(19) United States
(12) Patent Application Publication
(10) Pub. No.: US 2026/0355445 A1
(43) Pub. Date: Nov. 12, 2026

(54) INTEGRATED SYSTEM AND METHOD FOR AUTOMATED OXYGEN THERAPY MANAGEMENT WITH REMOTE MONITORING AND CONTROL

(71) Applicant: Carrie HOWARTH, Colorado Springs, CO (US)

(72) Inventor: Carrie HOWARTH, Colorado Springs, CO (US)

(21) Appl. No.: 19/725,709

(22) Filed: Jun. 30, 2026

Related U.S. Application Data
(63) Continuation of application No. PCT/US2026/017622, filed on Mar. 4, 2026.
(60) Provisional application No. 63/766,895, filed on Mar. 4, 2025.

(51) Int. Cl. A61M 16/00; A61B 5/00; A61B 5/1455

(52) U.S. Cl. CPC A61M 16/024; A61B 5/0004; A61B 5/14552; A61B 5/4839; A61B 5/6826; A61B 5/742; A61M 2016/0033; A61M 2202/0208; A61M 2205/3334; A61M 2205/3584; A61M 2205/3592; A61M 2205/502

(57) ABSTRACT

A universal retrofittable system for automated oxygen therapy management comprises a flow control hardware device connecting as an add-on between existing oxygen sources and patient oxygen lines to regulate flow rates from 0-20 liters per minute without requiring source modifications. An integrated oximeter device combines blood oxygen and heart rate monitoring with direct manual control functionality through a finger-mounted interface, enabling zero-latency adjustment. A base unit co-houses flow control hardware and automated control software with touchscreen interface supporting manual and automatic mode switching. Wireless communication enables battery-powered oximeter telemetry via Bluetooth Low Energy protocols. A failsafe bypass valve ensures continuous oxygen availability during system deactivation. The system integrates with hospital management systems through standardized HL7 and FHIR protocols, enabling comprehensive monitoring from clinical workstations. Adaptive learning algorithms analyze historical oxygen requirement patterns to proactively adjust flow rates, while configurable threshold-based automation triggers adjustments when SpO2 measurements fall outside predetermined ranges. The architecture accommodates both standalone concentrators and medical gas outlets while maintaining consistent functionality across diverse care environments and concentrator models, providing a universal hardware abstraction layer for legacy oxygen infrastructure.

### CROSS REFERENCE TO RELATED APPLICATIONS

This application is a bypass continuation of International Patent Application No. PCT/US2026/017622, filed Mar. 4, 2026, entitled “INTEGRATED SYSTEM AND METHOD FOR AUTOMATED OXYGEN THERAPY MANAGEMENT WITH REMOTE MONITORING AND CONTROL,” which claims the benefit of U.S. Provisional Patent Application No. 63/766,895, filed Mar. 4, 2025, entitled “INTEGRATED SYSTEM AND METHOD FOR AUTOMATED OXYGEN THERAPY MANAGEMENT WITH REMOTE MONITORING AND CONTROL.” The entire contents of each of the foregoing applications are incorporated herein by reference.

### BACKGROUND OF THE INVENTION

Many individuals require supplemental oxygen therapy to maintain adequate oxygen levels, particularly in home healthcare environments and clinical settings managing chronic respiratory conditions. These patients often face significant challenges in managing their oxygen needs effectively and maintaining their independence. The need for supplemental oxygen therapy extends beyond chronic obstructive pulmonary disease (COPD) and other traditional respiratory conditions, encompassing various medical conditions including sleep apnea, interstitial lung disease, pulmonary hypertension, pulmonary fibrosis, congestive heart failure, cystic fibrosis, and post-operative recovery, all of which require careful monitoring and management of oxygen delivery. The patient population requiring oxygen therapy spans diverse age groups from pediatric patients with congenital conditions to elderly patients with age-related decline, diverse severity levels from mild exertional desaturation requiring supplementation only during activities to severe resting hypoxemia requiring continuous high-flow support, and diverse care settings from independent living in private residences to assisted living facilities, skilled nursing facilities, rehabilitation centers, and acute care hospitals.

A critical issue arises when patients need to move around within their living space or hospital room. While oxygen concentrators typically allow for movement using extended cannula tubing within a structure—commonly 50-foot cannula cords enabling movement throughout typical residential layouts with dimensions from 800 to 2,500 square feet, or 25-foot cords for more compact living spaces, or 100-foot cords for larger homes or facilities—even short walks of 15-20 feet can result in dangerous drops in oxygen concentration due to the physiological demands of physical exertion. For example, a patient's oxygen saturation (SpO2) may start at 96% or 98% when seated close to the concentrator in a resting state with minimal metabolic demand, but can drop precipitously to 84%, 82%, or even lower levels during movement spanning distances as short as 15 feet to a bathroom or 30 feet to a kitchen, despite medical guidance from pulmonologists and respiratory therapists that oxygen concentration should not fall below 90% for extended periods and ideally should remain above 92-94% to maintain adequate tissue oxygenation and prevent cellular hypoxia. This phenomenon, which may be termed the “Exertion-Desaturation Gap,” represents a fundamental challenge in oxygen therapy management: the static flow rate set on the oxygen source fails to address the dynamic oxygen demands imposed by patient movement and physical activity, creating a mismatch between oxygen delivery and oxygen consumption that worsens proportionally with activity intensity.

This problem is particularly acute because physical exertion, even from simple activities of daily living like walking to the restroom covering distances of 10-30 feet, standing to prepare meals requiring 5-20 minutes of upright posture, climbing stairs involving 5-20 steps with substantial elevation gain, bending to retrieve items from low shelves or floor level, or performing personal hygiene tasks requiring sustained physical effort, requires increased oxygen flow to maintain safe oxygen levels due to elevated metabolic demand where oxygen consumption can increase by factors of 1.5× to 5× compared to resting levels depending on activity intensity. When seated and at rest, a patient may maintain adequate oxygen levels with SpO2 readings of 94-98% indicating healthy oxygen saturation with a flow rate of 2, 4, or 6 liters per minute depending on disease severity and individual physiology. However, upon standing and walking, the SpO2 readings can drop rapidly within timeframes of 30 seconds to 3 minutes to 88%, 86%, 84%, or lower and continue declining due to the increased oxygen consumption from physical exertion combined with the unchanged oxygen flow rate creating an oxygen deficit that accumulates over time. The severity of this desaturation increases with the intensity and duration of physical activity, creating dangerous situations where patients experience respiratory distress including dyspnea, increased breathing effort, chest tightness, lightheadedness, or anxiety precisely when they are furthest from their oxygen source and least able to make necessary adjustments due to being engaged in activities, being in locations distant from the concentrator such as bathrooms or bedrooms at opposite ends of homes, or experiencing physical and cognitive impairment from the desaturation itself limiting problem-solving capability and physical coordination required to return to the concentrator and adjust settings.

The situation is further complicated by the current limitations of oxygen concentrator control mechanisms and their physical positioning. Patients often require assistance from others to adjust their oxygen flow rates, as the concentrator may be located far from where they need to be—in another room such as a utility room, laundry room, or closet chosen for noise isolation or aesthetic reasons, on a different floor level requiring stair climbing to access in multi-story residences, or positioned in a utility area for noise reduction or to minimize visible medical equipment in living spaces affecting home aesthetics and patient dignity. When oxygen levels drop during physical activity, flow rates typically need to be increased substantially, often from resting rates of 2-4 liters to active rates of 6, 7, 8 liters or more representing increases of 50% to 300%, to restore and maintain safe SpO2 readings of 94-98% or at minimum above the critical threshold of 90% below which tissue hypoxia and organ dysfunction risks increase substantially. However, many patients are reluctant to inconvenience family members who may be working, sleeping, or engaged in their own activities, caregivers who have multiple responsibilities and limited availability, or healthcare staff in clinical facilities who are managing multiple patients and responding to competing demands for assistance with these adjustments, particularly when adjustments may be needed multiple times daily in response to varying activity levels including morning routines, meal preparation, bathing, ambulation for exercise or therapy, and nighttime bathroom visits, creating adjustment frequencies ranging from 3-4 times daily for relatively sedentary patients to 10-20 times daily or more for active patients attempting to maintain independence and quality of life.

This reluctance to seek help for flow adjustment can lead to extended recovery times ranging from 5 to 30 minutes or longer as patients wait for their oxygen levels to slowly improve while remaining stationary, sitting or lying down to minimize metabolic demand, practicing controlled breathing techniques, or simply enduring desaturation until passive recovery occurs. More seriously, prolonged desaturation increases risk of stroke due to reduced cerebral oxygenation and potential thrombotic events, cardiac events including arrhythmias, myocardial ischemia, or acute decompensation in patients with underlying cardiac disease, acute respiratory failure where compensatory mechanisms become exhausted leading to ventilatory failure requiring emergency intervention, and cumulative organ damage affecting kidneys, liver, and other vital organs when oxygen levels remain below the required threshold of 90% for what medical professionals define as clinically significant duration. Medical professionals, including pulmonologists specializing in chronic respiratory disease management and respiratory therapists providing direct patient care and equipment management, often recommend emergency intervention through activation of emergency medical services (911), contact of on-call physicians, or presentation to emergency departments if oxygen levels remain in the 86-88% range for extended periods typically defined as 5-10 minutes without improvement despite attempts to rest and recover, or if levels fall below 85% at any time indicating severe hypoxemia, as sustained hypoxemia causes cellular damage at the mitochondrial level impairing ATP production, triggers inflammatory cascades, and can precipitate acute decompensation in patients with compromised cardiopulmonary reserve who lack physiologic capacity to tolerate additional stress.

A significant challenge in the current oxygen therapy landscape is the wide variation in oxygen concentrator models and their control interfaces across different manufacturers. Like the diversity seen in television remote controls before industry standardization efforts undertaken during the 1990s and 2000s establishing universal remote protocols, oxygen concentrators lack standardization across different manufacturers including major vendors such as Philips Respironics, Inogen, Invacare, Drive DeVilbiss, AirSep, ResMed, SeQual, Inova Labs, and numerous others and models within manufacturer product lines ranging from basic entry-level devices to advanced clinical-grade systems, making universal control solutions difficult to implement without custom adaptation to each device's proprietary control interface, communication protocol, or mechanical configuration. This inconsistency among concentrator models creates barriers for patients who need to manage multiple devices—such as when transitioning between home and clinical settings where different equipment is installed, when equipment is replaced due to maintenance needs, recalls, or upgrades requiring patient retraining on new interfaces, when using backup concentrators from different manufacturers as redundancy against primary device failure, or when receiving care from multiple providers using different equipment types reflecting their purchasing contracts or equipment preferences—or when receiving care from multiple providers using different equipment types. The lack of standardized control interfaces means that patients and caregivers must learn and adapt to different control mechanisms which may include rotary dials with varying scale markings and rotation directions, push-button interfaces with different button layouts and labeling conventions, touchscreen displays with manufacturer-specific menu structures and navigation patterns, or remote controls with proprietary button mappings and pairing procedures, adding complexity to oxygen therapy management and potentially impacting the quality of care through user confusion, adjustment errors, or delayed response to desaturation events and patient safety through increased risk of incorrect settings, failure to adjust when needed due to interface unfamiliarity, or equipment misuse.

Existing solutions addressing these challenges have proven inadequate or economically inaccessible for many patients. Specialized automated oxygen titration systems such as O2matic manufactured by Chromogear (Denmark) or FreeO2 manufactured by Oxynov (Canada) provide closed-loop control maintaining SpO2 within target ranges through continuous monitoring and automated flow adjustment algorithms, but these are expensive standalone units requiring replacement of functional existing concentrators that may have substantial remaining useful life at costs often exceeding $5,000-$10,000 per complete system including the automated titration device and compatible oxygen source, or requiring purchase of the automated titration device as an add-on at costs of $3,000-$7,000 while still requiring compatible oxygen sources from specific manufacturers, creating economic barriers particularly for home healthcare patients on fixed incomes including retirees relying on Social Security or pension income, disabled individuals with limited earning capacity, or patients in developing regions where healthcare budgets are constrained. Manufacturer-specific remote controls exist for some premium concentrator models such as the Inogen Rove offering wireless control for Inogen concentrators or the Philips SimplyGo Mini with optional remote capability but lack interoperability, functioning only with specific models from single manufacturers and becoming obsolete when equipment is upgraded to newer models even from the same manufacturer if control protocols change or replaced with devices from different manufacturers due to insurance coverage, equipment availability, or provider preference, requiring purchase of new remote controls, loss of remote control functionality, or continued use of incompatible equipment combinations. Passive monitoring systems where pulse oximeters transmit data to tablets, smartphones, or dedicated displays through Bluetooth or WiFi connections provide awareness of desaturation enabling patients or caregivers to observe SpO2 trends and receive alerts when levels fall outside target ranges, but do not provide direct control over the oxygen source, requiring patients to interact with secondary devices such as approaching the concentrator, manually adjusting flow dials or buttons, and returning to their location or seek assistance while experiencing respiratory distress—precisely when such interactions are most difficult due to dyspnea, physical exhaustion, cognitive impairment from hypoxemia, or distance from the oxygen source. The lack of a universal, retrofittable solution for remote oxygen control creates a significant barrier to patient independence limiting ability to live alone safely, maintain employment requiring reliable oxygen management, or engage in social activities, and safety as delayed adjustment to desaturation increases risk of falls, acute medical events, or progressive disease exacerbation.

### SUMMARY OF THE INVENTION

The present invention in an embodiment provides a universal retrofittable add-on system for remote control and automated management of oxygen flow delivery to patients from existing oxygen sources without requiring source modification, internal component replacement, or manufacturer-specific adaptation. The system comprises multiple coordinated components that work together to monitor oxygen levels through continuous physiological measurement and regulate oxygen delivery through precision flow control while enhancing patient independence by eliminating the need for physical access to oxygen sources and safety by responding rapidly to desaturation events, enabling patients to maintain optimal oxygen saturation during movement and physical exertion without requiring caregiver assistance or physical access to oxygen concentrators or wall outlets. The universal nature of the system is characterized by compatibility with diverse oxygen sources including stationary home concentrators rated for flows from 1-10 LPM from manufacturers such as Philips Respironics, Inogen, Invacare, and others, portable concentrators, high-output clinical concentrators rated for flows up to 20 LPM, hospital wall outlets connected to central medical gas systems, compressed gas cylinders with regulators, and liquid oxygen systems, standardized connection interfaces using medical gas fittings such as DISS, CGA, and ISO connectors eliminating the need for source-specific adapters, agnostic operation independent of concentrator control protocols or internal architectures, and non-invasive installation preserving existing equipment warranties and enabling removal without permanent modification to oxygen sources.

A key component is a flow control hardware device that connects as a universal add-on between an oxygen source and the patient's oxygen line through standardized medical gas fittings including threaded connections, quick-connect adapters, compression fittings, or barbed hose fittings enabling tool-free or simple wrench-based installation without specialized equipment or technical expertise. This hardware precisely regulates oxygen flow from maximum source output typically 5-12 liters per minute for home concentrators, 15 liters per minute for standard hospital outlets, or 20-25 liters per minute for high-flow sources down to specific flow rates required by the patient ranging from 0.5 LPM during rest to 15-20 LPM during exertion or acute desaturation through electronically-controlled proportional valves rated for 0-20 liters per minute, 0-25 liters per minute, or 0-30 liters per minute continuous operation providing sufficient capacity to accommodate typical therapeutic ranges with headroom for peak demands. The flow control hardware incorporates a failsafe bypass valve ensuring continuous oxygen availability during system deactivation or power loss, with the bypass valve automatically opening to enable manual flow control directly at the oxygen source when electronic control is unavailable due to power failure, system shutdown, component malfunction detected through self-diagnostics, or user preference for manual operation, ensuring that device failure or removal never interrupts oxygen delivery. The failsafe mechanism may employ normally-open valve designs where spring mechanisms hold the valve open and electrical power is required to close the valve, redundant valve pathways with automatic switchover upon primary path failure, or mechanical bypass routes activated by loss of electrical power or control signals. The system is designed to work with both standalone oxygen concentrators from diverse manufacturers operating on different internal control principles including pulse-dose systems delivering oxygen boluses synchronized to inhalation, continuous-flow systems providing steady oxygen streams, or dual-mode systems offering both delivery methods and standard medical gas outlets in clinical settings connected to centralized pipeline systems, portable cylinder manifolds, or liquid oxygen reservoirs, providing a universal hardware abstraction layer for legacy oxygen infrastructure that presents a consistent control interface to users and automated systems regardless of underlying oxygen source type, manufacturer, or vintage.

The invention in an embodiment incorporates an integrated oximeter device that combines oxygen monitoring with remote control functionality through a wireless finger-mounted unit, toe-mounted unit, earlobe clip, forehead sensor, or other physiological monitoring location. This device continuously monitors blood oxygen concentration measured as SpO2 percentage and heart rate measured in beats per minute while enabling both automated and manual control of oxygen flow rates through a built-in interface accessible directly at the patient's fingertip or other sensor location. The wireless oximeter transmits physiological telemetry to a base unit via Bluetooth Low Energy protocols operating in the 2.4 GHz ISM band with transmission ranges from 30 to 300 feet depending on implementation and environmental factors, WiFi protocols providing longer range and higher bandwidth, proprietary sub-GHz protocols offering superior building penetration, or other wireless protocols, enabling patient mobility throughout living spaces spanning dimensions from studio apartments of 400 square feet to large homes of 5,000 square feet or more, multi-story residences with vertical separation up to 30 feet between floors, clinical facility rooms and adjacent corridors, or outdoor areas within wireless range such as patios, porches, or yards while maintaining continuous monitoring and control capability without tethering to bedside monitors or requiring line-of-sight to base units. When oxygen levels fall below predetermined thresholds configured by healthcare providers or patients based on clinical recommendations ranging from 85% to 95% for lower thresholds and 95% to 100% for upper thresholds, the system automatically triggers flow rate adjustments through the flow control hardware increasing flow by preset increments such as 0.5, 1.0, or 2.0 LPM, proportional amounts based on desaturation severity, or adaptive amounts learned from patient response patterns. The integrated control interface within the oximeter housing provides zero-latency manual override capability through physical buttons, capacitive touch sensors, touchscreen interfaces, or voice command recognition, enabling patients to adjust their oxygen delivery immediately upon experiencing respiratory distress, anticipating increased oxygen demand before activities such as climbing stairs or walking outdoors, or preferring manual control during specific activities such as sleep or exercise without requiring access to separate control devices such as smartphones that may not be carried at all times, tablets that may be located in other rooms, or the oxygen source which may be positioned in inaccessible locations such as closets, basements, or garages.

In an exemplary embodiment optimized for initial deployment and clinical validation, the flow control hardware and automated control software are co-housed within a base unit chassis comprising an integrated touchscreen interface providing direct user interaction without requiring external devices. The touchscreen provides manual/automatic mode switching enabling users to toggle between autonomous algorithmic control where the system adjusts flow rates automatically based on SpO2 measurements and direct manual flow rate specification where users select desired flow rates independent of measurements, while displaying real-time physiological telemetry including SpO2 updated at rates from 0.5 to 5 Hz, heart rate displayed in beats per minute, and current oxygen delivery rate shown in liters per minute with resolution from integer values to tenths or hundredths of a liter. Additional display information may include alarm status with color-coded indicators, wireless connectivity strength to the oximeter, battery level of the wireless oximeter unit, trending data showing SpO2 variations over selectable time windows from minutes to hours, or historical logs of flow adjustments and alarm events. This configuration simplifies communication architecture by enabling direct wired connections between co-located flow control and processing components through circuit board traces, ribbon cables, wire harnesses, or backplane connectors, eliminating wireless protocol requirements between the regulation mechanisms including proportional valves, flow sensors, pressure transducers, and safety bypass valves and the control processor including microcontrollers, digital signal processors, or embedded computing platforms executing control algorithms and user interface software while maintaining wireless connectivity to the remote oximeter device for physiological monitoring. The co-housed architecture reduces component count by sharing power supplies, enclosures, mounting hardware, and connection interfaces, simplifies installation requiring mounting and connection of only a single unit rather than multiple distributed components with intervening cable runs, enhances reliability by eliminating communication links between critical components as potential failure points, and facilitates troubleshooting and maintenance by consolidating all serviceable components at a single accessible location.

The system's intelligence is driven by automated control software that continuously processes oximeter readings sampled at rates of at least once per second, twice per second, or higher frequencies up to 5-10 Hz and executes precise flow adjustments through the proportional valve hardware with adjustment resolution from 0.01 to 0.5 LPM and response times from 0.5 to 5 seconds. The software implements threshold-based control algorithms comparing measured SpO2 against configurable limits including lower thresholds typically ranging from 85% to 95% and upper thresholds typically ranging from 95% to 100%, automatically increasing flow rates when desaturation is detected with SpO2 falling below lower thresholds and decreasing rates when adequate oxygenation is maintained with SpO2 above lower thresholds or exceeding upper thresholds, thereby optimizing oxygen delivery based on real-time patient needs and minimizing unnecessary oxygen consumption which reduces concentrator wear, power consumption, and operating costs. The software may include adaptive learning capabilities analyzing historical oxygen requirement patterns over time periods from days to months to proactively adjust flow rates during activities associated with desaturation risk such as morning routines, meal times, bathing, or specific exercises, predictive algorithms using machine learning models to anticipate desaturation before it occurs based on preceding physiological patterns such as increasing heart rate or decreasing perfusion index, or personalized control profiles customized to individual patient response characteristics rather than generic population-based algorithms. Data logging functionality maintains detailed therapy logs at fixed sampling rates capturing all system parameters including flow rate, SpO2, heart rate, operating mode, alarm status, and timestamp information with timestamps accurate to the second or millisecond, enabling healthcare providers and patients to review complete time-series records of oxygen level variations and flow rate modifications over time periods from hours to weeks to months for purposes including treatment optimization, medication titration decisions, activity counseling, equipment performance assessment, or clinical research. The system implements configurable alarm generation activating audible and visual notifications when oxygen levels remain outside desired ranges despite automated flow adjustments for durations exceeding persistence requirements ranging from 15 seconds to 5 minutes, with alarm persistence requirements filtering transient measurement artifacts from motion, sensor displacement, or environmental interference while ensuring timely alerts for sustained dangerous desaturation indicating genuine physiological deterioration or system malfunction requiring intervention. Healthcare providers configure treatment parameters including threshold values based on clinical assessment and treatment goals, maximum flow limits constrained by equipment capacity and prescription parameters, alarm conditions tailored to patient acuity and risk profiles, and operating modes appropriate for patient capability and care setting through the touchscreen interface in initial implementations or, in subsequent product iterations, through mobile applications running on iOS or Android devices, desktop applications on Windows or Mac computers, web-based portals accessible through standard browsers, or hospital EHR integration receiving configuration parameters from physician order entry systems and transmitting therapy data to clinical documentation systems using HL7 or FHIR protocols.

### BRIEF DESCRIPTION OF THE FIGURES

FIG. 1 illustrates a system overview in an embodiment showing an oxygen concentrator with an attachable remote control mechanism for controlling oxygen flow.

FIG. 2 shows an oximeter device in an embodiment with integrated remote control functionality that can be placed on a user's finger to monitor oxygen concentration and heart rate.

FIG. 3 depicts a finger-worn oximeter comprising a user interface configured to control the oxygen flow of the oxygen line to the wearer of the oximeter in accordance with an embodiment.

FIG. 4 shows the communication system architecture in accordance with an exemplary embodiment between the in-line flow control device and mobile phone/remote control mechanisms, and in an example including connectivity to medical records and oxygen control systems.

FIG. 5 illustrates an exemplary usage in accordance with an embodiment depicting the in-line flow control device placed upon an oxygen line originating form a wall, for instance in a hospital setting, communicatively linked to an automated control software interface to facilitate the activation of the in-line flow control device to facilitate the flow of oxygen.

FIG. 6 shows a flowchart illustrating the automated control process for oxygen flow regulation in accordance with an exemplary embodiment.

### DETAILED DESCRIPTION

In a preferred embodiment of the invention, the system provides an integrated solution for remote control and automated management of oxygen flow from oxygen concentrators. The system comprises multiple interconnected components that work together to monitor and regulate oxygen delivery while providing patients with enhanced independence and safety in managing their oxygen therapy.

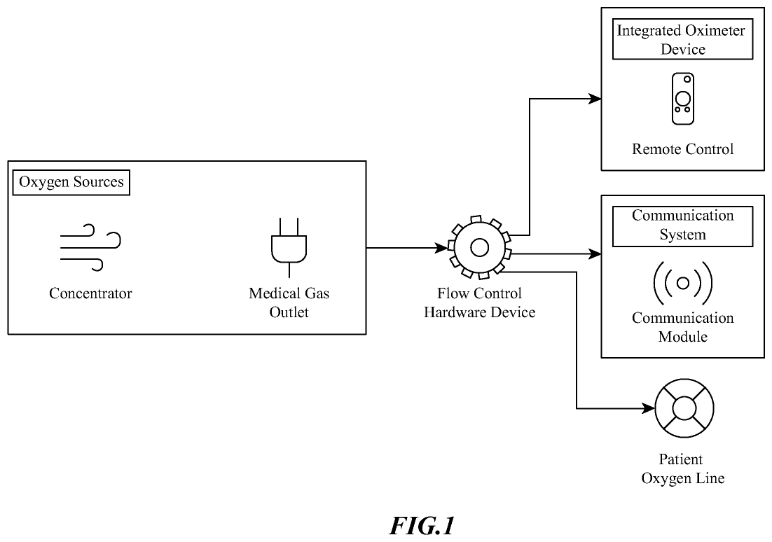

FIG. 1 illustrates a system overview in an embodiment showing an oxygen concentrator with an attachable remote control mechanism for controlling oxygen flow, wherein the universal add-on architecture enables retrofittable attachment to existing concentrator models from diverse manufacturers without requiring internal modification of concentrator components, disassembly of concentrator housings, alteration of concentrator control circuits, or voiding of manufacturer warranties, and wherein the system comprises a base unit co-housing flow control hardware including proportional valves and flow sensors and automated control software executing on embedded processors communicatively linked to a wireless oximeter device through Bluetooth Low Energy, WiFi, or other wireless protocols providing bidirectional data exchange including physiological telemetry transmission from oximeter to base unit and control command transmission from base unit to oximeter.

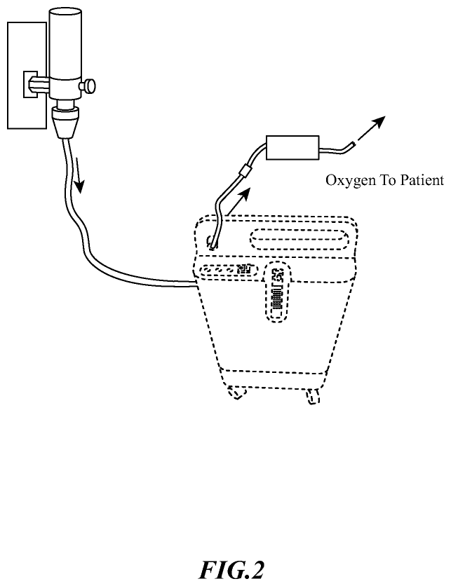

FIG. 2 shows an oximeter device in an embodiment with integrated remote control functionality that can be placed on a user's finger to monitor oxygen concentration and heart rate, wherein the oximeter device comprises a battery-powered wireless communication unit incorporating rechargeable lithium-ion or lithium-polymer batteries with capacities from 100 to 500 milliamp-hours transmitting physiological telemetry including SpO2 percentage, heart rate in beats per minute, perfusion index, and signal quality metrics to the base unit via Bluetooth Low Energy protocols operating at transmission intervals from 0.5 to 10 seconds with automatic reconnection upon signal interruption, and wherein the integrated remote control functionality enables zero-latency manual flow rate adjustments through user interface elements including physical buttons, capacitive touch sensors, or touchscreen displays incorporated directly within the oximeter housing enabling patients to increase or decrease flow rates, toggle between manual and automatic modes, or silence alarms without requiring access to the base unit, smartphones, or oxygen source.

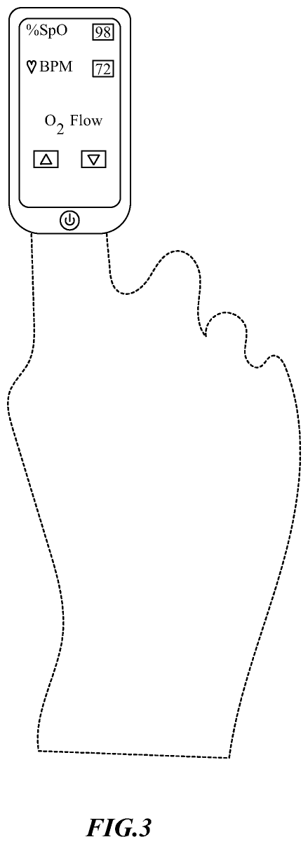

FIG. 3 depicts a finger-worn oximeter comprising a user interface configured to control the oxygen flow of the oxygen line to the wearer of the oximeter in accordance with an embodiment, wherein the user interface comprises physical buttons including increment and decrement controls or capacitive touch sensors enabling slide gestures or tap gestures enabling direct patient interaction with flow control mechanisms for manual adjustment of oxygen delivery rates, and wherein the oximeter continuously transmits oxygen saturation and pulse rate measurements sampled at rates of at least once per second to the flow control hardware device enabling both automated threshold-based adjustments where the system automatically increases flow when SpO2 falls below configured lower thresholds such as 88%, 90%, or 92% and decreases flow when SpO2 exceeds configured upper thresholds such as 96%, 98%, or 100% and manual user-directed adjustments where patients directly command flow rate changes through oximeter interface interaction overriding automated control temporarily or persistently based on user preference or clinical protocols.

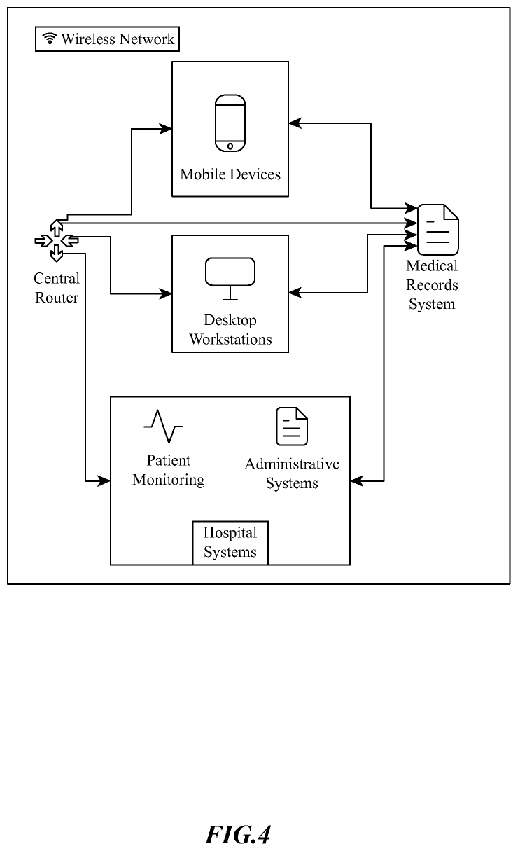

FIG. 4 shows the communication system architecture in accordance with an exemplary embodiment between the in-line flow control device and mobile phone/remote control mechanisms, and in an example including connectivity to medical records and oxygen control systems through standardized healthcare communication protocols including HL7 Version 2.x messaging for ADT feeds and result reporting and FHIR APIs for structured data exchange with electronic health record systems, wherein the communication architecture supports multiple control interfaces operating concurrently including integrated oximeter controls providing fingertip-accessible adjustment, touchscreen base unit interface providing local configuration and monitoring, mobile applications on smartphones or tablets providing remote monitoring within homes or from distant locations via internet connectivity, and hospital clinical workstation access enabling healthcare providers to monitor multiple patients simultaneously, adjust parameters remotely, and document interventions in clinical records, with arbitration mechanisms resolving conflicting commands through priority hierarchies, timestamp ordering, or clinical override authorities.

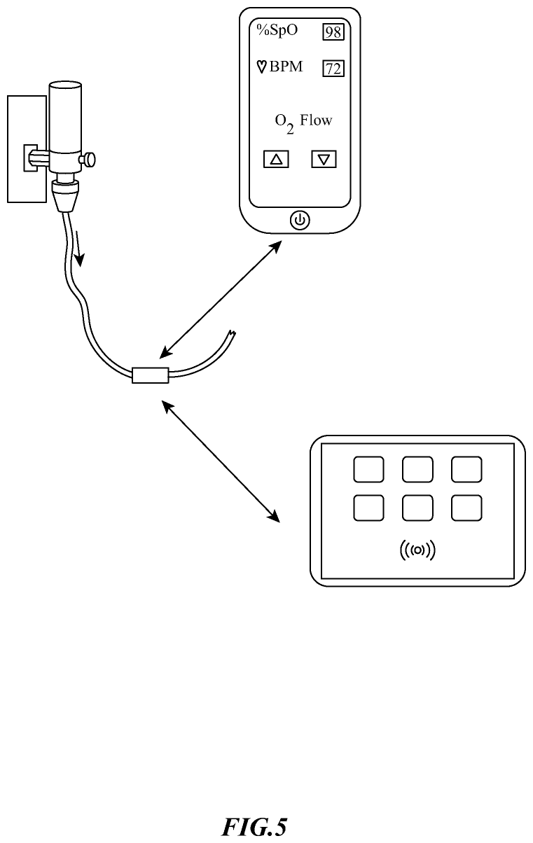

FIG. 5 illustrates an exemplary usage in accordance with an embodiment depicting the in-line flow control device placed upon an oxygen line originating from a wall outlet, for instance in a hospital setting, communicatively linked to an automated control software interface to facilitate the activation of the in-line flow control device to regulate the flow of oxygen, wherein the universal add-on configuration enables identical functionality whether the oxygen source comprises a standalone concentrator positioned in a home utility room or bedroom or a medical gas outlet mounted in a hospital room wall or ceiling-mounted service column connected to centralized pipeline systems operating at 50-55 PSI, and wherein the failsafe bypass valve ensures uninterrupted oxygen availability during system deactivation through user shutdown command, power loss from electrical outage or circuit breaker tripping, or component failure detected through self-diagnostic routines by automatically opening a normally-open bypass path redirecting oxygen flow around electronic control elements and enabling manual flow control at the oxygen source.

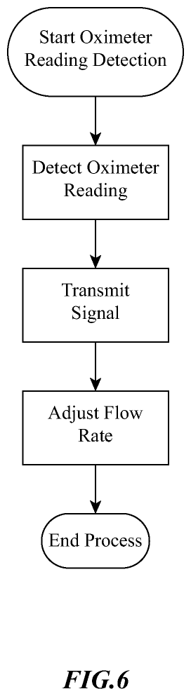

FIG. 6 shows a flowchart illustrating the automated control process for oxygen flow regulation in accordance with an exemplary embodiment, wherein the process comprises continuous monitoring of SpO2 measurements at sampling rates of at least once per second or higher frequencies up to 5 Hz providing temporal resolution sufficient to detect rapid desaturation events, threshold comparison with configurable upper and lower limits set by healthcare providers or patients based on clinical recommendations, automated flow rate adjustment commands transmitted to precision flow control hardware rated for 0-20 liters per minute regulation or higher capacities such as 0-25 or 0-30 LPM through proportional valves with positioning resolution from 0.01 to 0.5 LPM, verification of adjustment effectiveness through subsequent SpO2 monitoring observing whether desaturation resolves within expected timeframes from 30 seconds to 5 minutes, and detailed data logging of all adjustments with timestamps accurate to the second or millisecond enabling historical therapy pattern analysis including identification of desaturation triggers, assessment of automated control effectiveness, optimization of threshold parameters based on observed patient responses, and clinical documentation supporting treatment decisions and insurance reimbursement.

In an embodiment, the core component is a universal remote control mechanism that can be attached to existing oxygen concentrators. In the preferred embodiment of the invention, this remote control functionality is integrated into a finger oximeter device that continuously monitors both the patient's oxygen concentration and heart rate. The system can operate in either automated or manual mode, allowing for flexible control based on patient and caregiver preferences.

In an example of the invention, the hardware implementation includes a flow control component that connects directly to the oxygen line. This component comprises a permanently fixed oxygen line that connects to the concentrator on one end and provides an output for oxygen flow to the patient on the other. The flow control hardware can either interface directly with the concentrator where such connectivity is possible, or function as an independent regulation mechanism attached to the oxygen line.

In an embodiment, a communication system enables seamless interaction between all components. The flow control hardware receives signals from multiple possible interfaces including mobile devices, desktop applications, or dedicated remote controls. In a preferred embodiment, this communication infrastructure also supports integration with medical records systems and oxygen control systems, allowing for comprehensive monitoring and management of patient care.

In an example of the invention, the system's intelligence is driven by automated control software that continuously monitors oximeter readings and adjusts oxygen flow accordingly. When oxygen concentration drops below predetermined thresholds (such as 90), the system automatically increases flow rates to maintain safe oxygen levels. In a preferred embodiment, healthcare providers can configure these settings through mobile or desktop applications, making the system particularly suitable for both home and hospital environments.

In a preferred embodiment of the invention, the remote control mechanism comprises a system for wirelessly controlling oxygen flow through communication with a flow control hardware device. The remote control functionality can be implemented through multiple user interface options that communicate with the flow control hardware to regulate oxygen delivery.

In an embodiment, the system comprises a mobile application which provides users with an interface for oxygen flow control. This application establishes a wireless connection to the flow control hardware and enables users to monitor and adjust their oxygen flow rates in real-time. The mobile implementation offers an accessible solution for users already familiar with smartphone technology.

For enhanced accessibility, another embodiment implements the control interface as a wearable device application. This implementation, for example via a smartwatch, provides particularly convenient access for users with limited mobility, allowing them to make quick adjustments through simple touch commands directly from their wrist. The wearable solution proves especially valuable during physical activities when accessing a phone may be impractical.

In an alternative embodiment, the system includes a dedicated universal remote control device. Drawing inspiration from universal TV remote technology, this device can be programmed to communicate with the flow control hardware to regulate oxygen delivery. The universal remote features an ergonomic design with large, easily accessible buttons specifically engineered for users who may have difficulty with smaller touch interfaces.

The preferred embodiment incorporates a communicative linkage enabling control of oxygen flow through the flow control hardware. This linkage consists of communication protocols that enable secure transmission of control signals between the remote interface and the flow control hardware, while specialized software drivers interpret and execute flow adjustment commands with precision.

The flow control hardware connects directly to the oxygen concentrator and the oxygen line, managing the actual regulation of oxygen flow. When the oxygen concentrator is set to its maximum output (typically 10-12 liters), the flow control hardware can reduce this flow to the precise level specified by the remote control interface. For example, if a patient requires 8 liters of oxygen, the flow control hardware will reduce the concentrator's maximum output accordingly based on commands received through the remote control system.

The system in a preferred embodiment is specifically engineered to work with existing oxygen concentrators, providing a cost-effective solution that preserves functional equipment. The flow control hardware employs non-invasive connection methods that maintain the integrity of the original concentrator and oxygen line system, while control capabilities enable seamless operation across different concentrator models.

In a preferred embodiment of the invention, the integrated oximeter device incorporates remote control functionality directly within the finger-mounted unit. This integration allows users to control oxygen flow settings directly through the interface of the oximeter device while simultaneously monitoring their blood oxygen levels and heart rate. The unified control and monitoring solution provides enhanced convenience and immediate access to flow adjustment capabilities when the wearer is not immediately proximal to the concentrator.

The device continuously monitors the user's blood oxygen concentration and heart rate through standard pulse oximetry technology. In an example of the invention, the oximeter device automatically triggers oxygen flow adjustments through the flow control hardware based on measured blood oxygen levels. When readings decrease below a predetermined threshold (such as 90), the system automatically signals the flow control hardware to increase oxygen delivery. Conversely, when readings exceed an upper threshold, the system triggers a reduction in flow, optimizing oxygen delivery based on real-time physiological needs.

In an embodiment, the oximeter device supports both automated and manual control modes. The automated mode continuously monitors oxygen levels and adjusts flow rates without user intervention, particularly valuable during sleep or physical activity. The manual mode enables users to directly adjust their oxygen flow through the integrated interface, providing control when personal adjustment is preferred.

The preferred embodiment includes a user interface integrated into the oximeter device that displays current oxygen saturation levels, heart rate, and oxygen flow settings. This interface provides immediate feedback on physiological parameters while enabling direct control of oxygen delivery through easily accessible buttons or touch controls incorporated into the device housing.

In an example of the invention, the oximeter device maintains wireless communication with the flow control hardware, transmitting both automated adjustment signals and manual control commands. The system can be configured with customizable thresholds for automated control, allowing healthcare providers to set appropriate parameters for individual patient needs. For instance, when a patient's oxygen level drops from 96 to 84 during physical activity, the system automatically increases flow rates from 6 liters to 7-8 liters to maintain optimal oxygen saturation.

In a preferred embodiment of the invention, the flow control hardware comprises a specialized component that connects between the oxygen concentrator and the patient's oxygen line. This hardware component is designed to regulate the flow of oxygen from the concentrator, which is typically set to its maximum output of 10-11 liters, down to the specific flow rate required by the patient. In a preferred embodiment of the invention, the flow control hardware implements continuous flow regulation, providing constant and uninterrupted oxygen supply through the permanently fixed oxygen line. This continuous flow capability enables the system to maintain precise control over oxygen delivery while responding to both automated and manual adjustment commands through the integrated control mechanisms.

The system incorporates a permanently fixed oxygen line that connects directly to the oxygen concentrator's output. In an embodiment, this fixed connection ensures a stable and secure attachment to the concentrator, maintaining consistent flow regulation capabilities. The flow control hardware includes a separate output port that connects to the patient's oxygen line, creating a complete path for oxygen delivery while maintaining flow control.

In a preferred embodiment, the flow control hardware can establish a direct communicative link with compatible oxygen concentrators. This direct linkage enables more precise control over oxygen delivery through digital communication with the concentrator's internal systems. However, the system is designed to function effectively even without direct concentrator communication, operating as an independent flow regulation mechanism that physically controls the oxygen flow through the line.

The flow control hardware incorporates precision mechanisms for regulating oxygen flow rates. In an example of the invention, when receiving signals from the remote control system, the hardware adjusts internal components to modify the flow rate. For instance, if a patient requires a reduction from the concentrator's maximum output of 10-11 liters down to 6 liters while at rest, the flow control hardware precisely regulates the flow to maintain this specified rate.

In an embodiment of the invention, the flow control hardware is designed to integrate with standard medical gas outlets found in hospital room settings and other clinical environments. The system connects to wall-mounted oxygen supply ports through medical-grade fittings and adapters, enabling the same precise flow control capabilities available with standalone concentrators.

The flow control hardware in this embodiment includes specialized connection interfaces that are compatible with standardized medical gas outlet specifications used in hospital environments. This allows the system to regulate oxygen flow from the building's central oxygen supply system, while maintaining communication with the hospital's monitoring and control systems through the integrated communication infrastructure.

In an example of the invention, the flow control hardware connects between the wall-mounted oxygen outlet and the patient's oxygen delivery line in a hospital environment. When installed in this configuration, the system maintains its ability to receive control signals from remote interfaces and automated monitoring systems, adjusting the oxygen flow rate from the building's supply system in response to patient needs or clinical protocols. The hardware regulates the higher-pressure hospital oxygen supply down to appropriate therapeutic flow rates, similar to how it manages flow from standalone concentrators.

The system's communication capabilities in this embodiment enable integration with hospital management systems and electronic medical records, allowing staff to monitor and control oxygen delivery from nursing stations and other clinical workstations. This integration maintains consistent functionality whether the oxygen source is a wall-mounted medical gas outlet or a standalone concentrator, providing flexibility for different care environments while ensuring precise control over oxygen delivery.

In an embodiment, the flow control hardware includes integrated communication systems that enable it to receive signals from various control interfaces, including the integrated oximeter device, mobile applications, desktop applications, or dedicated remote controls. These signals direct the hardware to make precise adjustments to oxygen flow rates based on either manual commands or automated responses to the patient's oxygen levels.

The integrated communication system incorporates both hardware and software components to enable reliable signal transmission and processing. In an example of the invention, the hardware components include a wireless communication module supporting multiple protocols such as Bluetooth, WiFi, and cellular connectivity to ensure consistent communication with various control interfaces. The system also includes signal processing hardware that converts received commands into precise flow control adjustments.

The software architecture in a preferred embodiment comprises multiple layers for signal handling and flow control. A base communication layer manages wireless protocols and signal reception, while a middleware layer processes and validates incoming commands from the various control interfaces. The control software then interprets these commands and translates them into specific flow rate adjustments. For example, when receiving a signal from the oximeter device indicating low oxygen levels, the software automatically calculates the required flow rate increase and directs the flow control mechanisms accordingly.

In an embodiment, the system includes integration capabilities with medical records systems and oxygen control systems through standardized healthcare communication protocols. The software includes security features to ensure protected transmission of patient data and control signals, while maintaining compatibility with various healthcare facility systems. For instance, in a hospital setting, the desktop application can interface with the facility's electronic health records to log oxygen flow adjustments and patient oxygen levels over time.

In a preferred embodiment of the invention, the integrated communication system interfaces with hospital monitoring and management systems to enable comprehensive oxygen therapy control from multiple points of care. The system receives telemetry data from patient monitoring devices, such as bedside vital sign monitors, and integrates this information with the flow control system. For example, when patient monitoring systems detect changes in vital signs or oxygen saturation levels, this data is automatically processed through the hospital's electronic medical records system and can trigger appropriate adjustments to oxygen flow rates via the flow control hardware.

The system enables authorized medical staff to monitor and control oxygen delivery from nursing stations and other clinical workstations throughout the facility. In an embodiment, the desktop application interface at nursing stations provides real-time monitoring of multiple patients' oxygen levels and flow rates, while allowing staff to make immediate adjustments when needed. The communication system processes commands from these workstations through the hospital's secure network infrastructure to the flow control hardware, enabling rapid response to changing patient conditions.

In an example of the invention, the system integrates with hospital management systems to coordinate oxygen therapy with other aspects of patient care. When changes in patient status are recorded in the electronic medical records system—such as during pre-operative preparation or post-operative recovery—the system can automatically adjust oxygen flow rates according to predetermined protocols. The flow control hardware receives these commands through the integrated communication system and implements the required flow adjustments, while maintaining a secure audit trail of all changes in the hospital's records system.

In a preferred embodiment of the invention, the communication system employs a multi-layered architecture to enable robust signal transmission between control interfaces and the flow control hardware. The system utilizes redundant communication pathways, ensuring reliable connectivity even in challenging healthcare environments where signal interference may be present.

The system implements specialized communication protocols designed for medical device integration. In an example of the invention, these protocols enable secure, low-latency transmission of control signals while maintaining medical-grade reliability standards. The communication system employs encryption and authentication mechanisms to protect patient data and ensure that only authorized devices and applications can control oxygen flow rates.

In an embodiment, the system includes a dedicated signal processing unit that manages multiple simultaneous connections from different control interfaces. This allows concurrent monitoring and control access from mobile devices, desktop applications, and dedicated remote controls, with intelligent arbitration to manage potentially conflicting commands. For instance, the system can prioritize automated safety adjustments from the oximeter device while still maintaining responsiveness to manual controls from healthcare staff.

The communication system incorporates standardized healthcare integration protocols to enable seamless connectivity with existing medical infrastructure. In a preferred embodiment, the system supports HL7 and FHIR protocols for medical records integration, allowing bi-directional data exchange with electronic health record systems. This enables automatic documentation of oxygen therapy adjustments while allowing the system to respond to care protocols initiated through the hospital's management systems.

In an example of the invention, the communication system includes a gateway component that manages the interface between local device control and broader healthcare systems. This gateway handles protocol translation, ensuring that control signals from mobile applications and remote devices are properly formatted for the flow control hardware, while simultaneously managing the aggregation and transmission of therapy data to medical records systems.

The system supports dynamic device pairing and authentication to facilitate secure connections between control interfaces and the flow control hardware. In an embodiment, this includes automated discovery and configuration capabilities that simplify the process of connecting new control devices while maintaining security. For example, when a new mobile device needs to be authorized for control, the system manages the secure exchange of credentials and establishes encrypted communication channels to protect patient data and control signals.

In a preferred embodiment of the invention, the automated control software provides intelligent management of oxygen flow rates based on real-time monitoring of patient oxygen levels. The software continuously processes data from the integrated oximeter device and executes precise flow rate adjustments through the flow control hardware to maintain optimal oxygen saturation.

The system in accordance with an embodiment implements threshold-based control algorithms. The associated thresholds in an example are controlled via adjustable settings in communicatively-linked user interfaces, in various examples via connected electronic health records systems or mobile applications operating on smart devices configured to control the system. In an example of the invention, when the oximeter detects that blood oxygen concentration has dropped below a predetermined threshold (such as 90), the software automatically triggers an increase in oxygen flow through the flow control hardware. For instance, if a patient's oxygen level drops from 96 to 84 during physical activity, the system will automatically increase the flow rate from 6 liters to 7-8 liters to restore proper oxygenation.

In a preferred embodiment, the software provides comprehensive configuration capabilities through both mobile and desktop applications. Healthcare providers can customize treatment parameters including threshold levels, flow rate ranges, and adjustment increments. The desktop application interface, particularly valuable in hospital settings, enables clinical staff to configure multiple patient profiles and establish automated response protocols based on individual care requirements. The system's automated control software and configuration interfaces accommodate the significant variations in oxygen therapy requirements between individual patients. Healthcare providers can customize flow parameters, threshold levels, and adjustment protocols through the mobile and desktop applications to match each patient's specific medical needs and circumstances. This configurability ensures optimal oxygen delivery across different conditions and activity levels while maintaining automated safety monitoring. The historical tracking capabilities provide essential documentation for treatment optimization, allowing healthcare providers to identify patterns in oxygen requirements and adjust therapeutic protocols accordingly. This data-driven approach enables more precise and personalized oxygen therapy management while maintaining comprehensive records of all flow rate modifications.

The control software incorporates adaptive learning capabilities in an embodiment of the invention. The system monitors patterns in oxygen requirements and adjusts its response parameters accordingly. For example, if a patient consistently requires increased flow during certain activities or times of day, the software can proactively adjust flow rates to prevent oxygen desaturation events.

In an example of the invention, the software maintains detailed logs of all automated adjustments and system operations. These logs integrate with medical records systems, providing healthcare providers with comprehensive therapy data for monitoring treatment efficacy and patient compliance. The system includes configurable alerts that can notify both patients and healthcare providers when significant adjustments are made or when oxygen levels remain outside desired ranges despite flow rate changes.

In a preferred embodiment of the invention, in accordance with an exemplary use the method for controlling oxygen delivery comprises continuous monitoring of the patient's blood oxygen concentration and heart rate through the integrated oximeter device. The device employs standard pulse oximetry technology to obtain real-time measurements of these vital parameters, which are essential for determining appropriate oxygen flow rates.

The system wirelessly transmits the measurement data from the oximeter device to the flow control hardware through secure communication protocols. In an example of the invention, this transmission occurs via multiple wireless protocols including Bluetooth, WiFi, and cellular connectivity to ensure reliable data delivery. The communication system's signal processing hardware converts these measurements into a format suitable for automated analysis.

The automated control software continuously compares the received measurements against predetermined threshold values configured by healthcare providers. In a preferred embodiment, when blood oxygen concentration falls below critical thresholds (such as 90), the system initiates automatic flow rate adjustments. For example, if readings drop from 96 to 84 during physical activity, the system triggers an increase in flow from 6 liters to 7-8 liters.

The flow control hardware executes precise adjustments to oxygen delivery based on these automated commands. In an embodiment, the hardware regulates flow from the maximum concentrator output (typically 10-12 liters) down to the specific rate determined necessary by the control system. The integrated oximeter interface simultaneously enables manual flow rate adjustments, allowing patients to directly control their oxygen delivery when desired.

In a hospital environment, the method incorporates additional steps for comprehensive care integration. The system receives telemetry data from various patient monitoring devices through the hospital's secure network infrastructure. This data is processed through the electronic medical records system using standardized healthcare protocols such as HL7 and FHIR, enabling automated flow adjustments based on the broader context of patient care.

The system maintains detailed logs of all flow rate modifications, whether initiated automatically or manually. In an example of the invention, these logs are automatically integrated with medical records systems, providing healthcare providers with comprehensive therapy data. The automated control software analyzes this data to generate configurable alerts when oxygen levels remain outside desired ranges despite flow adjustments, or when significant changes in oxygen requirements are detected.

The method includes intelligent alert generation based on continuous monitoring. When oxygen levels remain in concerning ranges (such as 86-88) for extended periods (5-10 minutes) despite flow adjustments, the system can trigger notifications to both patients and healthcare providers through multiple interfaces. In a preferred embodiment, these alerts are customizable through both mobile and desktop applications, allowing for personalized notification thresholds and delivery methods.

In a preferred embodiment of the invention, the integrated oxygen therapy management system combines multiple coordinated components to provide comprehensive monitoring and control of oxygen delivery. The system centers around a flow control hardware component that connects between the oxygen source-whether a standalone concentrator or hospital wall outlet and the patient's oxygen line. This hardware precisely regulates oxygen flow rates based on both automated and manual control inputs.

The flow control hardware receives control signals through an advanced communication system that enables interaction with multiple interface options. In the preferred embodiment, an integrated oximeter device incorporates remote control functionality directly into a finger-mounted unit, allowing patients to monitor their oxygen levels while controlling flow rates. The system also supports control through mobile applications, desktop interfaces, and dedicated remote controls, providing flexibility for different usage scenarios.

The automated control software serves as the system's intelligence, continuously monitoring oximeter readings and adjusting flow rates to maintain optimal oxygen levels. When blood oxygen concentration falls below predetermined thresholds, the software automatically signals the flow control hardware to increase oxygen delivery. This automation works in concert with manual controls, allowing healthcare providers to configure treatment parameters through mobile and desktop applications.

In hospital environments, the system integrates seamlessly with existing medical infrastructure. The communication system enables connectivity with electronic health records and hospital management systems, while the flow control hardware adapts to standard medical gas outlets. This integration allows for comprehensive monitoring and control from nursing stations and other clinical workstations, while maintaining detailed therapy records.

The coordinated operation of these components creates a comprehensive solution that addresses the challenges of oxygen therapy management. From automated flow adjustment based on patient needs to remote monitoring capabilities for healthcare providers, the system enhances both patient independence and treatment efficacy. The flexible architecture accommodates various care environments while maintaining consistent functionality, whether deployed in home healthcare settings or hospital facilities.

In accordance with an embodiment, the system architecture prioritizes core automated titration and local control functionality while maintaining extensibility for future feature integration. This implementation demonstrates how the universal retrofittable approach enables deployment of life-critical safety features using proven components and mature communication protocols, while the modular architecture preserves pathways for subsequent enhancement. The architecture may be implemented through various configurations including centralized designs where all processing occurs within a single base unit, distributed architectures where processing distributes across multiple interconnected modules, or hybrid approaches combining centralized critical control functions with distributed user interface and communication capabilities. The extensibility may be achieved through software-defined interfaces, standardized communication protocols, modular hardware connectors enabling component upgrades, or combinations thereof. In certain implementations, the system employs field-programmable gate arrays (FPGAs) or application-specific integrated circuits (ASICs) providing hardware acceleration for real-time control loops while maintaining software configurability for higher-level algorithms. Alternative embodiments utilize general-purpose microcontrollers or microprocessors executing firmware stored in flash memory, enabling field updates without hardware modifications.

In accordance with an embodiment, clinical validation and regulatory approval pathways benefit from focused feature sets addressing primary patient safety needs. Implementations may focus on fundamental challenges including enabling patients to maintain safe oxygen saturation during movement and physical exertion without requiring assistance from caregivers or physical access to the oxygen source, reducing caregiver burden through automated monitoring and adjustment, preventing hypoxemia-related complications through rapid response to desaturation events, or combinations of these objectives. By establishing core functionality through systems comprising co-housed base unit hardware with wireless oximeter telemetry, distributed systems with separate flow control and monitoring components, or integrated all-in-one devices incorporating all functions within a single housing, the invention delivers therapeutic value while demonstrating the universal add-on architecture's viability across diverse oxygen sources including stationary home concentrators, portable concentrators, hospital wall outlets, compressed gas cylinders, liquid oxygen systems, and patient environments ranging from private residences to skilled nursing facilities to acute care hospitals. The viability demonstration may include compatibility testing across concentrator models from manufacturers such as Philips Respironics, Inogen, Invacare, Drive DeVilbiss, AirSep, and others, verification of performance across varying supply pressures from 5 PSI to 60 PSI, testing under environmental conditions spanning temperature ranges from 50 °F to 95 °F and humidity levels from 15% to 95% relative humidity, and validation with patient populations including COPD, interstitial lung disease, pulmonary fibrosis, congestive heart failure, and post-operative recovery patients.

In accordance with an embodiment, the system implements touchscreen-based configuration and control at the base unit, mobile application interfaces on smartphones or tablets, desktop software applications on personal computers or workstations, web-based interfaces accessible through standard browsers, voice-controlled interfaces responding to spoken commands, or combinations of multiple interface modalities providing redundant access methods. This design approach offers advantages including standalone operation requiring no additional computing devices, simplified system setup through guided configuration wizards, reduced potential points of failure by eliminating dependencies on external devices, universal accessibility not requiring smartphone ownership or technical proficiency, and immediate tactile feedback through direct touch interaction. Users may access configuration parameters including threshold values ranging from 70% to 100% SpO2 for lower limits and 85% to 100% for upper limits, maximum flow rate limits from 1 to 20 liters per minute, alarm delay periods from 5 seconds to 10 minutes, hysteresis bands from 0.5% to 5% SpO2, adjustment step sizes from 0.1 to 2.0 LPM per adjustment cycle, and maximum adjustment velocities from 0.5 to 5.0 LPM per minute. Real-time physiological telemetry displayed may include SpO2 percentage updated at rates from 0.5 Hz to 5 Hz, heart rate in beats per minute with precision from integer values to tenths of a beat, perfusion index indicating signal quality, plethysmograph waveforms showing pulse characteristics, respiration rate derived from pulse variability analysis, and trending data showing variations over selectable time windows from 1 minute to 24 hours. Logged therapy data accessible through the interface may encompass complete time-series records sampled at rates from once per second to once per ten seconds, event logs capturing threshold violations and alarm activations, adjustment histories documenting every flow rate change with timestamps precise to the second or millisecond, usage statistics showing cumulative therapy hours and compliance metrics, and battery history tracking charge cycles and capacity degradation over the device lifetime.

In accordance with an embodiment, the base unit touchscreen interface displays a manual/automatic mode selector, a semi-automatic mode offering automated suggestions requiring user confirmation before implementation, a scheduled mode executing predefined flow rate profiles based on time of day or activity calendars, a learn mode observing user manual adjustments to develop personalized automated profiles, or combinations enabling mode switching based on user preferences or clinical protocols. The interface may enable users to toggle between modes through simple touch gestures such as tapping mode icons, swiping between mode screens, using dedicated hardware buttons adjacent to the touchscreen, voice commands when speech recognition is enabled, or automatic mode transitions triggered by detected conditions such as alarm states or communication failures. In automatic mode, the system executes threshold-based control algorithms, predictive algorithms using machine learning models trained on historical patient data, adaptive algorithms that modify thresholds dynamically based on observed patterns, fuzzy logic controllers providing smooth transitions between control states, proportional-integral-derivative (PID) controllers minimizing SpO2 error while avoiding overshoot, model predictive controllers optimizing flow trajectories over future time horizons, or hybrid approaches combining multiple algorithmic strategies. The algorithms continuously adjust flow rates to maintain SpO2 within configured ranges which may be fixed ranges such as 92%-96%, activity-dependent ranges that widen during exercise and narrow during rest, time-of-day dependent ranges addressing circadian variations in oxygen requirements, position-dependent ranges accounting for supine versus upright posture effects, or dynamically calculated ranges based on trending analysis of recent measurements. In manual mode, users directly specify desired flow rates through touchscreen controls including numeric keypad entry for precise value input, slider controls enabling continuous adjustment across the available range, increment/decrement buttons for stepwise changes in predetermined increments from 0.1 to 1.0 LPM, preset buttons providing one-touch access to frequently used flow rates such as 2, 4, 6, and 8 LPM, or quick-adjustment gestures such as swipe-up to increase or swipe-down to decrease by preset amounts. The system maintains the specified rate regardless of SpO2 measurements, or alternatively, provides advisory notifications when SpO2 deviates from safe ranges while respecting the manual mode selection, or implements safety override mechanisms that temporarily increase flow despite manual mode when SpO2 falls below critical thresholds such as 85% for longer than emergency timeout periods such as 2-5 minutes. Manual mode proves particularly valuable during activities where patients have learned through experience their oxygen requirements, such as specific exercise routines including treadmill walking, stationary cycling, or resistance training, sleeping positions including supine, prone, or elevated head positions, respiratory exercises such as pursed-lip breathing or diaphragmatic breathing, eating and swallowing which may temporarily require increased flow, bathing and personal care activities, or travel situations where patients proactively adjust settings based on altitude changes or anticipated exertion levels, allowing them to proactively set appropriate flow rates rather than reactively responding to desaturation events.

In accordance with an embodiment, the touchscreen displays numerical flow rate information showing current oxygen delivery measured in liters per minute with resolution from integer values to hundredths of a liter, SpO2 percentage displayed as integer values or with decimal precision to tenths of a percent, heart rate in beats per minute as integer values or with fractional beat precision, respiration rate in breaths per minute when derivable from pulse variability or through separate respiration sensors, wireless connectivity status including signal strength indicators showing bars, percentage, or received signal strength indicator (RSSI) values in dBm, connection state indicators distinguishing between connected, connecting, disconnected, and pairing states, and link quality metrics such as packet loss rates or latency measurements. Battery level information for the wireless oximeter unit may be displayed as percentage remaining from 0-100%, estimated runtime remaining in hours and minutes, charge state indicators showing charging, fully charged, or discharging, battery health indicators showing capacity relative to design specification, and historical battery performance data tracking charge cycles and degradation trends. Alarm status indicators may include active alarm conditions with visual highlighting through color coding including red for critical alarms, yellow for warnings, and green for normal operation, textual descriptions specifying the alarm condition such as “SpO2 Below Threshold” or “Weak Oximeter Signal,” alarm priority levels distinguishing between informational notices, caution alerts, warnings, and critical alarms, time since alarm activation showing elapsed duration, and alarm history showing recently cleared alarms with timestamps. Threshold configuration controls accessible through the touchscreen menu system, setup wizards, quick-access widgets on the main screen, or gesture-activated pop-up interfaces enable users or healthcare providers to set upper and lower oxygen saturation limits that trigger automated intervention, wherein the limits may be configured as single threshold values such as 90% for lower limit and 98% for upper limit, dual threshold systems with warning thresholds at 91% and critical thresholds at 88% providing graduated response, asymmetric thresholds with tighter lower bounds and wider upper bounds reflecting different risk profiles, dynamically adjustable thresholds that modify based on trending data, activity-specific thresholds stored as profiles selectable by activity type, or time-scheduled thresholds that change automatically based on time of day corresponding to sleep, rest, or activity periods. For example, in accordance with specific clinical scenarios, a pulmonologist may configure a lower threshold of 88%, 90%, or 92% SpO2 for a COPD patient with specific activity-related desaturation patterns documented through prior monitoring, while setting an upper threshold of 96%, 98%, or 100% to prevent hyperoxia during rest periods, reflecting clinical evidence regarding optimal oxygenation targets for specific patient populations. Alternative configurations may establish lower thresholds at 85% for patients with chronic hypoxemia tolerance, at 93% for patients requiring tight glycemic control where oxygenation affects metabolic stability, or at 95% for post-operative patients requiring elevated oxygenation during healing phases.

In accordance with an embodiment, flow rate adjustments—whether automated based on oximeter readings, manually initiated via touchscreen controls, triggered by schedule-based protocols, or initiated through remote caregiver intervention—are logged at sampling rates that capture system parameters. The data logging subsystem may sample and record system state information at fixed rates including once per second, twice per second, once every two seconds, once every five seconds, once every ten seconds, or at adaptive rates that increase during periods of instability and decrease during stable periods to optimize storage utilization. Recorded parameters may include current flow rate with precision from 0.1 to 0.01 LPM, SpO2 measurement as integer percentage or with decimal precision, heart rate in beats per minute, perfusion index indicating signal quality on scales from 0-20% or 0-100%, pulse waveform amplitude data, operating mode indicators specifying manual, automatic, semi-automatic, scheduled, or learn mode status, alarm condition flags indicating active alarms or recent alarm history, timestamp information with precision from one-second resolution to millisecond resolution, source pressure measurements when available from concentrator telemetry or integrated pressure sensors, ambient temperature and humidity when environmental sensors are incorporated, patient activity level when accelerometer data is available from the oximeter or separate wearable sensors, GPS location data when mobile applications with location services are integrated, or caregiver interaction events documenting user interface operations. This complete data capture creates therapy records enabling healthcare providers to analyze oxygen requirement patterns over time scales from hours to days to weeks to months, identify correlations between activities and desaturation events through time-series analysis, pattern recognition algorithms, or machine learning classification models, optimize threshold settings through simulation of historical data with candidate threshold configurations, optimize maximum flow rate limits based on empirical patient-specific data showing actual flow requirements during various activities, predict equipment maintenance needs through analysis of valve cycling frequency and pressure variations, assess patient compliance through detection of manual overrides or system deactivation periods, evaluate treatment efficacy through trending of average SpO2 levels and desaturation event frequency over treatment courses, or support clinical research through de-identified data aggregation across patient populations.

In accordance with an embodiment, the logging approach may employ fixed sampling rate methodologies recording all parameters at regular intervals, event-based logging systems that record data only when changes occur or thresholds are crossed, hybrid approaches combining fixed-rate baseline logging with increased sampling during events, compressed storage techniques using run-length encoding for stable periods or differential encoding storing only changes from previous values, cloud-based logging transmitting data to remote servers for unlimited storage and advanced analytics, local storage with periodic synchronization to external devices or cloud services, or distributed logging maintaining separate logs on multiple system components with subsequent merging during analysis. While event-based logging reduces storage requirements by factors ranging from 2× to 100× depending on data stability, the fixed-rate approach provides complete time-series data preserving information about stability periods and gradual trends that event-based systems might miss. For example, in accordance with a specific clinical scenario, a gradual decline in SpO2 from 95% to 91% over time periods ranging from 5 minutes to 30 minutes might not trigger event logging at any individual measurement if event thresholds are set at 90% or below, but the time-series record clearly shows the trend enabling early intervention before the patient experiences symptomatic desaturation, predicts future desaturation through extrapolation or trend analysis algorithms, identifies underlying causes through correlation with concurrent parameter changes such as increasing heart rate or decreasing perfusion index, or triggers preventive flow increases before SpO2 reaches alarm thresholds through predictive algorithms. Alternative embodiments implement sophisticated event detection analyzing rate of change metrics such as SpO2 declining faster than 0.5%/minute, 1%/minute, or 2%/minute, acceleration metrics detecting when the rate of decline is itself increasing, pattern matching algorithms identifying characteristic desaturation signatures associated with specific activities or conditions, or multivariate anomaly detection analyzing combinations of parameters to identify clinically significant events that single-parameter thresholds would miss.

In accordance with an embodiment, logged data is stored in non-volatile memory within the base unit, on removable storage media such as SD cards or USB flash drives, in cloud storage services accessible via internet connectivity, on connected mobile devices through application caching, or in distributed fashion across multiple storage locations providing redundancy. Storage capacity may be sufficient for periods ranging from 24 hours to 30 days or more of continuous second-by-second logging depending on data compression ratios, parameter count, and storage precision. In certain implementations, the system maintains rolling buffers storing the most recent 7 days, 14 days, 30 days, 60 days, or 90 days of therapy data, with automatic overwriting of oldest records when storage capacity is reached, archival of old data to secondary storage before overwriting, selective retention of clinically significant events and alarm periods while summarizing stable periods, or dynamic storage management that extends retention by reducing sampling rates for older data. Users may export logged data through multiple mechanisms including USB connections to the base unit enabling file transfer to personal computers or USB flash drives, wireless transmission via Bluetooth or WiFi to paired mobile devices, email attachments sent through cloud services when internet connectivity is available, generation of QR codes encoding summarized data for scanning into electronic medical records systems, or direct transmission to healthcare provider portals through secure healthcare information exchanges. Exported data formats may include comma-separated values (CSV) files compatible with spreadsheet applications such as Microsoft Excel, Google Sheets, or LibreOffice Calc, JavaScript Object Notation (JSON) files for web application consumption and API integration, Extensible Markup Language (XML) formatted according to healthcare data standards, HL7 Version 2.x messages or FHIR resources for electronic medical record integration, PDF reports with graphical visualizations and summary statistics suitable for clinical documentation, or proprietary binary formats optimized for space efficiency with companion viewer applications. Healthcare providers reviewing exported data gain insights including average SpO2 levels over various time periods, percentage of time spent within target SpO2 ranges, frequency and duration of desaturation events classified by severity, flow rate utilization patterns showing distribution across available range, alarm event frequency and response times, circadian patterns in oxygen requirements, activity-related patterns correlating SpO2 changes with patient-reported or sensor-detected activities, treatment adherence metrics showing system usage hours and manual override frequency, equipment performance metrics identifying sensor issues or connectivity problems, or longitudinal trends comparing current performance against historical baselines that inform treatment optimization decisions including threshold adjustments, medication titration, pulmonary rehabilitation referrals, or equipment upgrades.

In accordance with an embodiment, the system includes configurable alarm conditions that activate when oxygen saturation levels fall outside predetermined thresholds for duration periods, when heart rate exceeds or falls below configurable limits, when flow rate reaches maximum limits indicating insufficient capacity, when wireless connectivity is lost for extended periods, when battery levels become critically low, when sensor signal quality degrades indicating improper placement or equipment malfunction, or when combinations of conditions indicate complex clinical scenarios requiring intervention. Unlike instantaneous threshold violations that might result from motion artifacts during patient movement, transient sensor displacement when adjusting finger position, environmental interference from fluorescent lighting or electromagnetic sources, or measurement noise inherent to photoplethysmography, the alarm system implements persistence requirements, signal quality gating, multivariate confirmation, or combinations thereof to reduce false alarms. Persistence requirements may specify that SpO2 must remain continuously below threshold values such as 85%, 88%, or 90% for consecutive time periods ranging from 15 seconds to 5 minutes before alarm activation, that a certain percentage such as 80%, 90%, or 95% of measurements within a sliding time window must fall below threshold, that median or average SpO2 calculated over windows from 30 seconds to 2 minutes must fall below threshold eliminating outlier effects, or that specific numbers such as 10, 20, or 30 consecutive measurements below threshold must occur. Signal quality gating may inhibit alarm activation when perfusion index falls below threshold values such as 0.5%, 1.0%, or 2.0% indicating inadequate signal, when pulse waveform variability exceeds limits indicating motion artifact, when signal-to-noise ratios fall below specifications, or when oximeter reports error conditions. Multivariate confirmation may require simultaneous conditions such as low SpO2 accompanied by elevated heart rate suggesting physiologic desaturation rather than measurement artifact, low SpO2 accompanied by high flow rate indicating genuine oxygen insufficiency rather than system malfunction, or patterns across multiple parameters matching clinical profiles of specific emergency conditions. For example, in accordance with specific configurations, an alarm may be configured to activate only when SpO2 remains below 88% for 60 consecutive seconds while signal quality remains adequate, filtering out brief measurement excursions from finger movement, coughing, or sensor repositioning while still alerting users to sustained dangerous desaturation requiring intervention. Alternative configurations may use shorter persistence periods such as 15-30 seconds for critically ill patients requiring rapid response, or longer periods such as 2-5 minutes for stable patients where temporary desaturation during activities like stair climbing is expected and tolerable.

In accordance with an embodiment, when alarm conditions are met, the system activates notification mechanisms that may include audible alarms from base unit speakers, visual indicators on touchscreen displays, haptic feedback through vibration motors in wireless oximeters or mobile devices, remote notifications transmitted to caregiver smartphones or tablets, automated messages to healthcare provider on-call systems or nurse call stations in clinical facilities, activation of external alarm devices such as bed shakers for hearing-impaired patients or strobe lights for noisy environments, or combinations providing redundant notification through multiple sensory channels and communication paths. Audible alarms may sound from base unit speakers at volume levels configurable from 50 to 100 decibels, with selectable tones including continuous tones, pulsed tones with frequencies from 0.5 to 10 Hz, rising or falling frequency sweeps, melodic alarm patterns, spoken voice alerts announcing specific alarm conditions through text-to-speech synthesis, or multi-tone patterns where different tones indicate different alarm priorities or types. Volume may be user-adjustable through touchscreen controls, automatically adjusted based on ambient noise levels detected through microphones, scheduled to reduce during nighttime hours, or boosted automatically during alarm escalation. Visual alarm indicators appearing on touchscreen displays may employ high-contrast color coding including red backgrounds for critical alarms requiring immediate action, orange or yellow backgrounds for warning conditions requiring attention, blue backgrounds for informational alerts, or flashing colors alternating between alarm colors and normal backgrounds at frequencies from 0.5 to 3 Hz to draw attention. Textual descriptions of alarm conditions may include simple labels such as “Low Oxygen,” more detailed descriptions such as “SpO2 Below 88% for 2 Minutes,” clinical guidance such as “Check Cannula Placement and Contact Provider,” or dynamic messages incorporating current measurements such as “Current SpO2: 86%—Target Range: 92-96%.” Alarm displays may show elapsed time since alarm activation, countdown timers until alarm escalation, buttons for alarm acknowledgment or temporary silencing, and historical alarm logs showing recent cleared alarms with timestamps and durations. The alarms may persist until explicitly acknowledged by users through touchscreen interaction such as tapping acknowledge buttons, swiping to dismiss, entering PIN codes preventing accidental dismissal, or using dedicated physical buttons, or may auto-clear when the alarm condition resolves for specified periods such as 30 seconds to 2 minutes ensuring stability before clearance, with optional requirements for manual confirmation even after auto-clear. This ensures that alarms cannot be missed due to temporary absence from the immediate vicinity of the base unit, sleeping through initial alarms, cognitive impairment preventing alarm recognition, or environmental noise masking audible alerts.

In accordance with an embodiment, the alarm system differentiates between conditions addressed by automated flow adjustments and conditions requiring user intervention through analysis of system response effectiveness, flow rate utilization, trending data, or combinations thereof. When automated control successfully responds to desaturation by increasing flow rates and SpO2 subsequently returns to target range within response periods from 30 seconds to 5 minutes, the system may log the event in therapy records with classifications such as “Automated Correction” or “Successful Intervention” but not activate alarms, recognizing that automation performed its intended function, or may provide subtle notifications through non-alarming visual indicators or brief tones acknowledging the automated response for user awareness. However, when SpO2 remains depressed below threshold levels despite automated flow rate increases reaching configured maximum flow limits, persisting for durations from 1 minute to 10 minutes, trending downward despite maximum flow indicating worsening condition, or accompanied by other concerning parameters such as very high heart rate, the system activates alarms with priority levels escalated based on severity, indicating that automated intervention has proven insufficient and user or caregiver action is needed. Required actions communicated through alarm messages, textual guidance on displays, or pre-recorded voice instructions may include verifying proper cannula placement in nostrils and checking for kinking or obstruction, checking oxygen source supply levels including concentrator power and operation indicators or cylinder pressure gauges, increasing manual flow rate settings at the source beyond system maximum if source capacity permits, switching to backup oxygen source if available, contacting medical support through emergency services, on-call providers, or telehealth consultations, or administering emergency medications such as bronchodilators when prescribed protocols permit. Additional alarm conditions requiring user intervention may include equipment malfunctions detected through self-diagnostics such as valve failures, sensor failures, or communication failures, power supply issues such as AC power loss when backup batteries are depleted or low, consumable depletion such as oxygen concentrator filters requiring replacement, or environmental hazards such as detected oxygen leaks when oxygen concentration sensors are incorporated.

In accordance with certain embodiments, alarm configurations include escalation protocols activating additional notification mechanisms if initial alarms are not acknowledged within timeframes ranging from 1 minute to 30 minutes depending on alarm severity and patient clinical status. For example, if initial audible alarms sound continuously or in repeated cycles for periods such as 5 minutes, 10 minutes, or 15 minutes without user acknowledgment detected through touchscreen interaction, button presses, or oximeter control inputs, the system may escalate by increasing alarm volume by increments from 10 to 30 decibels, changing alarm tones to more attention-demanding patterns such as sirens or rapid beeping, activating additional alarm outputs such as flashing lights or external alarm devices connected via wired or wireless interfaces, sending automated notification messages through multiple communication channels including SMS text messages to stored caregiver phone numbers, push notifications to mobile applications on caregiver devices, emails to configured addresses with alarm details and patient status, automated voice calls to emergency contacts with text-to-speech announcements, or transmissions to healthcare provider messaging systems, medical alert monitoring services, or emergency response dispatch systems. Escalation may proceed through multiple stages with progressively more aggressive interventions, such as stage 1 (immediate) activating local alarms, stage 2 (after 5 minutes) notifying local caregivers via mobile devices, stage 3 (after 15 minutes) notifying healthcare providers or monitoring services, and stage 4 (after 30 minutes) initiating emergency services contact. This escalation approach provides additional safety layers for patients who might be incapacitated due to severe desaturation causing confusion or loss of consciousness, sleeping deeply and not awakening to initial alarms, cognitively impaired and unable to recognize alarm significance, hearing impaired and missing audible alerts, or physically unable to respond due to mobility limitations or concurrent medical emergencies. Alternative escalation strategies may employ severity-based escalation where critical alarms immediately notify caregivers while less urgent warnings escalate gradually, location-based escalation that first attempts to contact caregivers known to be home before notifying remote contacts, time-of-day based escalation with different protocols for daytime versus nighttime hours, or patient-customized escalation reflecting individual risk profiles and available support systems. Certain implementations provide user configuration of escalation parameters through setup interfaces, allowing patients and families to define contact priorities, notification methods, time delays, and escalation trees matching their specific care arrangements.

In accordance with an embodiment, the wireless oximeter configuration comprises a battery-powered wireless communication unit, a wired unit with low-voltage power delivery through lightweight cables, an ultra-low-power optical sensor with energy harvesting from ambient light, or a hybrid approach with battery backup and opportunistic charging through the base unit. The wireless implementation configured for finger mounting, toe mounting, earlobe mounting, forehead mounting using reflectance oximetry, wrist mounting with peripheral pulse detection, or other body locations enables patients to move freely throughout environments including typical residential spaces ranging from 500 to 5,000 square feet, multi-story homes with vertical separation up to 30 feet, clinical facility rooms and adjacent corridors covering ranges from 50 to 200 feet from base unit, outdoor areas within wireless range such as patios, porches, or gardens, or mobile scenarios such as walking neighborhoods with portable base units while maintaining continuous physiological monitoring and bidirectional communication with the stationary or mobile base unit containing flow control hardware and automated control software. Unlike wired pulse oximeters that tether patients to bedside monitors with cable lengths from 3 to 15 feet severely limiting mobility, or standalone pulse oximeters lacking control integration and requiring patients to manually check readings and separately adjust oxygen sources, this wireless implementation employs communication protocols providing ranges from 30 feet (10 meters) for basic Bluetooth configurations to 300 feet (100 meters) for Bluetooth long-range extensions or WiFi-based implementations, with range extending techniques including mesh networking where multiple base units relay signals, strategic placement of wireless repeaters or range extenders throughout homes, directional antennas focusing radio energy toward frequently occupied areas, or automatic power adjustment maintaining minimum necessary transmission power to preserve battery life while ensuring reliable connectivity.

In accordance with an embodiment, the wireless oximeter transmits SpO2 and heart rate measurements, pulse waveform data, perfusion index values, sensor status information, battery levels, or combinations thereof to the base unit via Bluetooth Low Energy (BLE) protocols, WiFi protocols such as 802.11b/g/n/ac operating in 2.4 GHz or 5 GHz bands, proprietary sub-GHz protocols using frequencies such as 433 MHz, 868 MHz, or 915 MHz providing superior building penetration and range, Zigbee or other mesh networking protocols enabling multi-hop communication through intermediate devices, or cellular protocols such as LTE-M or NB-IoT providing wide-area connectivity. BLE technology offers advantages including low power consumption with transmission currents from 5 to 15 milliamps enabling battery life extensions from days to weeks, sufficient data throughput ranging from 125 kilobits per second to 2 megabits per second for transmitting physiological measurements sampled at rates from 0.5 to 5 Hz along with metadata, widespread adoption ensuring component availability from multiple vendors at competitive costs and interoperability with smartphones, tablets, and computers enabling expanded system capabilities, and robust connection maintenance protocols that handle temporary signal interruptions gracefully through automatic reconnection, buffering of measurements during disconnection periods, or graceful degradation maintaining core functionality at reduced update rates. The BLE connection operates in the 2.4 GHz ISM band using frequency-hopping spread spectrum techniques that provide resistance to interference from other wireless devices commonly present in home and clinical environments including WiFi access points operating on overlapping frequencies, microwave ovens generating broadband interference, cordless phones, baby monitors, Bluetooth classic devices, and wireless video cameras by rapidly switching among 40 channels at rates from 160 to 3200 hops per second, employing adaptive frequency hopping that identifies and avoids congested channels, using forward error correction coding providing resilience to corrupted packets, implementing automatic repeat request (ARQ) protocols retransmitting lost packets, or combinations thereof maintaining reliable communication despite challenging electromagnetic environments. Alternative embodiments employ WiFi direct protocols eliminating the need for intermediate access points, ZigBee protocols offering mesh networking where the oximeter can communicate through intermediate nodes such as other medical devices or home automation equipment, or ultra-wideband (UWB) technology providing precise indoor positioning data in addition to communication for tracking patient location and detecting falls or unusual movement patterns.

In accordance with an embodiment, the wireless oximeter unit incorporates rechargeable batteries including lithium-ion cells with capacities from 100 to 500 milliamp-hours, lithium-polymer cells offering flexible form factors, nickel-metal hydride cells providing cost advantages, or solid-state batteries providing enhanced safety, non-rechargeable primary batteries such as alkaline or lithium cells for extended shelf life in infrequently used devices, supercapacitors providing rapid charging and extended cycle life for applications with frequent charging opportunities, or hybrid systems combining batteries with energy harvesting from body heat through thermoelectric generators, motion through kinetic energy harvesting, or ambient light through photovoltaic cells. Operational runtime may be sufficient for extended use between charging cycles ranging from 8 hours for high-power continuous transmission configurations to 168 hours (7 days) for ultra-low-power configurations with optimized duty cycling, with actual runtime depending on transmission power levels, sampling rates, display brightness when local displays are incorporated, and environmental conditions affecting battery performance such as temperature extremes. In certain implementations, the battery capacity enables 24-48 hours of continuous operation including constant wireless transmission at intervals from 1 to 10 seconds, LED operation for optical sensing with drive currents from 5 to 50 milliamps, signal processing through microcontrollers consuming 1 to 10 milliwatts in active mode and microamps in sleep mode, and optional features such as vibration alerts, backlight displays, or haptic feedback. The oximeter may include charging interfaces such as USB-C ports supporting standard USB charging protocols at currents from 0.5 to 3 amperes enabling charge times from 30 minutes to 3 hours, micro-USB ports for backward compatibility, magnetic charging connectors eliminating wear on mechanical contacts and providing automatic alignment, wireless inductive charging through Qi-compatible charging pads or custom inductive couplers, conductive charging cradles with spring-loaded contacts, or solar charging panels for outdoor use scenarios enabling overnight charging, opportunistic charging during rest periods, or rapid top-up charging during meal breaks while the patient sleeps or engages in sedentary activities, ensuring full charge availability for the following day's use, maintaining minimum charge thresholds above 20-30% to maximize battery cycle life, or supporting continuous operation when charging infrastructure is available such as in clinical facilities or during nighttime rest.

In accordance with an embodiment, battery charge status information is transmitted wirelessly to the base unit and displayed on the touchscreen interface, shown on local displays integrated into the oximeter housing, announced through audible voice alerts, communicated through LED indicator patterns with color coding such as green for charged, yellow for moderate charge, and red for low charge, or combinations thereof, enabling users to monitor remaining runtime and plan charging accordingly. Low battery warnings may appear on the touchscreen with textual messages, graphical battery icons showing depletion levels, color-coded warning bars, or animated indicators, and optionally trigger audible notifications including beeps, spoken announcements, or melodic tones when remaining charge falls below predetermined levels including 50%, 30%, 20%, 10%, or 5% capacity, providing advance notice ranging from 30 minutes to 12 hours to charge the device before battery depletion interrupts monitoring depending on power consumption rates and remaining capacity. In some embodiments, the oximeter unit includes local battery level indicators such as LED status lights with multiple colors indicating charge ranges, segmented LED displays showing charge level in quartiles or deciles, numeric percentage displays on small OLED or LCD screens, or dynamic indicators showing discharge rate predictions providing charge information visible directly on the oximeter without requiring wireless communication to the base unit, ensuring battery awareness even when wireless connectivity is temporarily lost or the base unit is powered off.

In accordance with an embodiment, the wireless oximeter maintains continuous transmission of physiological data to the base unit throughout normal operation, periodic transmission at intervals optimized for battery conservation while maintaining adequate temporal resolution for clinical monitoring, event-triggered transmission sending data when significant changes occur, or adaptive transmission adjusting update rates based on detected patient state with higher rates during unstable periods and lower rates during stable rest. Continuous transmission implementations used in certain configurations remain actively transmitting from the moment they detect proper finger placement through capacitive sensing, optical perfusion detection, or mechanical pressure switches until removed from the finger detected through loss of pulse signal, absence of capacitive coupling, or timeout periods, ensuring that the base unit receives real-time physiological data enabling immediate automated response to desaturation events occurring within seconds, contrasting with periodic measurement approaches sampling at intervals from 10 seconds to several minutes that might introduce delays between desaturation onset and system response potentially allowing SpO2 to decline further before intervention. Alternative implementations employ intelligent transmission strategies that increase update rates from baseline values such as once per 10 seconds to rapid rates such as twice per second when SpO2 trends downward, heart rate becomes erratic, perfusion index decreases, or alarm conditions approach, balancing battery conservation during stable periods with responsive monitoring during critical events.

In accordance with an embodiment, the wireless communication link between the oximeter and base unit implements automatic connection management protocols including device discovery, pairing, authentication, encryption negotiation, and connection establishment. When the system powers on, the devices automatically establish wireless pairing through Bluetooth device discovery broadcasting identifiers and capabilities, authentication using pre-shared keys, numeric comparison, or passkey entry, and channel establishment negotiating communication parameters. Once paired initially, the devices remember pairing credentials stored in non-volatile memory and automatically reconnect when brought within communication range detected through periodic advertising packets or connection attempts, eliminating the need for repeated pairing procedures during normal daily use. If the wireless connection is temporarily interrupted—for example, if the patient moves to a location experiencing signal attenuation from intervening walls, metal structures, or water-filled obstacles, electromagnetic interference from nearby equipment, or exceeds the maximum communication range—the devices continuously attempt reconnection through repeated connection requests at intervals from 0.5 seconds to 10 seconds, automatically reestablishing the link when conditions improve through patient movement, interference source removal, or environmental changes, with reconnection typically occurring within timeframes from 1 second to 30 seconds depending on protocol parameters and signal conditions.

In accordance with an embodiment, the communication system includes error detection and signal quality monitoring through multiple mechanisms operating at different protocol layers. Each transmitted data packet includes error detection codes such as checksums computed through simple summation algorithms, cyclic redundancy check (CRC) values using polynomials such as CRC-16 or CRC-32 providing stronger error detection, or forward error correction (FEC) codes such as Reed-Solomon or convolutional codes enabling correction of bit errors without retransmission, enabling the receiving base unit to verify data integrity by recomputing the error detection code and comparing against the received value. Invalid packets with mismatched error codes are discarded, with the system awaiting the next valid transmission arriving within typical intervals from 1 to 10 seconds rather than acting on corrupted data that might indicate false desaturation or erroneous flow adjustment commands, or requesting retransmission through automatic repeat request (ARQ) protocols when transmission reliability is prioritized over latency. The oximeter firmware monitors optical signal quality metrics including perfusion index calculated from the ratio of pulsatile to non-pulsatile signal components with typical values ranging from 0.3% to 20%, signal-to-noise ratio comparing pulse amplitude to baseline noise with acceptable ratios above 10:1, 20:1, or higher thresholds, pulse waveform consistency evaluating shape regularity and detecting irregular patterns indicating motion artifact or arrhythmias, and light sensor saturation detecting excessive ambient light or LED drive levels, transmitting signal quality indicators along with SpO2 and heart rate values as confidence metrics, quality flags, or numerical scores. The base unit software uses these quality indicators to assess measurement reliability by comparing against acceptable quality thresholds, optionally inhibiting automated flow adjustments when signal quality falls below acceptable thresholds preventing inappropriate automation responses to artifact-contaminated measurements showing falsely low SpO2 values, displaying quality warnings alerting users to reposition sensors or address signal issues, or logging quality metrics for subsequent analysis of measurement reliability and sensor performance trends.

In accordance with certain embodiments, the wireless oximeter includes local user interface elements providing immediate patient feedback without requiring line-of-sight to the base unit or dependency on wireless connectivity. These local interface elements may include LED status indicators with multiple colors such as red, yellow, and green showing operational states including powered on, actively measuring, wireless connected, battery charging, or alarm conditions, small display screens such as OLED panels with dimensions from 0.5 to 2 inches diagonal showing current SpO2 and heart rate values updated in real-time, segmented numeric displays showing SpO2 as two-digit or three-digit values, graphical indicators showing trend arrows indicating whether SpO2 is stable, rising, or falling, wireless connectivity status icons showing signal strength or connection state, battery level icons or percentage displays, or text messages providing brief status information. These local indicators might display current SpO2 values with numerical precision from integer percentages to tenths of a percent, heart rate values in beats per minute, timestamps showing duration of current measurement session, historical minimum or maximum values reached during the session, wireless connectivity status distinguishing between strong signal, weak signal, or disconnected states, alarm conditions with color-coded indicators or textual labels, or operational mode indicators showing whether the system is in manual or automatic control mode. However, in certain phased implementations, the primary user interface remains the base unit touchscreen providing more extensive information display and control options, with the oximeter's compact form factor prioritizing comfortable wearability through minimized size and weight, extended battery life through reduced power consumption from displays and processing, durability through simplified mechanical design with fewer interface elements subject to wear, and clinical focus ensuring that physiological monitoring functions receive design priority over extensive local interface features that might increase device complexity, cost, or failure modes.

In accordance with an embodiment, the base unit architecture co-locates the flow control hardware and host controller components in a single integrated chassis, separates these components in distributed architectures with interconnection through wired or wireless links, or employs modular designs enabling field configuration of distributed or integrated topologies based on installation requirements. The integrated design approach embodied in certain implementations contrasts with distributed architectures where flow control mechanisms might be positioned at the oxygen source location such as a utility closet, garage, or medical equipment room while control processors and user interfaces are located in frequently occupied living spaces such as bedrooms or family rooms requiring wireless or wired communication links spanning distances from 10 to 100 feet. The co-housed configuration offers advantages including simplified communication architecture eliminating long-distance signal transmission challenges, reduced component count by sharing power supplies, enclosures, and connection interfaces, simplified installation requiring mounting and connection of only a single device rather than multiple distributed components with intervening cable runs or wireless configuration, enhanced reliability by eliminating connection points and communication links between flow control and control processing as potential failure modes, reduced system cost by consolidating components and eliminating redundant housings and interfaces, and simplified troubleshooting and maintenance with all critical components accessible at a single location.

In accordance with an embodiment, co-housing the flow control hardware and host controller within the base unit enables direct wired connections between these components through circuit board traces, ribbon cables, wire harnesses, or backplane connectors, eliminating wireless protocol requirements between the flow regulation mechanisms including proportional valves, flow sensors, and pressure transducers and the automated control processing unit including microcontrollers, FPGAs, or microprocessors executing control algorithms. Wired connections between co-located components provide benefits over wireless alternatives including zero latency beyond electrical propagation delays measured in nanoseconds to microseconds, eliminating wireless packet transmission delays ranging from milliseconds to tens of milliseconds and collision avoidance protocols introducing additional variable delays, immunity to radio frequency interference from other devices ensuring reliable command delivery with bit error rates below one error per billion bits transmitted even in electromagnetically noisy environments containing WiFi routers, cellular devices, motor drives, or RF medical equipment, deterministic timing enabling precise synchronization between sensor sampling and valve actuation critical for stable closed-loop control, simplified protocol stacks avoiding the overhead of wireless physical layer modulation, medium access control, error correction, and network management reducing processing requirements and power consumption, enhanced security by eliminating wireless eavesdropping or jamming attack surfaces, power delivery through wired connections eliminating battery requirements for flow control actuators enabling continuous operation without battery maintenance or the periodic charging cycles required for distributed wireless actuators, or combinations thereof. The wired interface may employ serial communication protocols such as UART, SPI, or I2C operating at rates from 9.6 kilobits per second to 10 megabits per second, parallel interfaces providing simultaneous multi-bit data transfer, analog control signals such as 0-10 volt or 4-20 milliamp current loops providing direct valve positioning commands, pulse-width modulation (PWM) signals encoding commands through duty cycle variation, or Controller Area Network (CAN) or similar automotive-grade protocols providing robust communication in noisy electrical environments.

In accordance with an embodiment, the base unit chassis comprises a rigid enclosure constructed from medical-grade plastics such as ABS, polycarbonate, or PEEK meeting biocompatibility standards including ISO 10993 and USP Class VI, coated metals such as powder-coated steel or anodized aluminum providing electromagnetic shielding and mechanical strength, composite materials combining structural supports with aesthetic covers, or combinations thereof providing mechanical protection for internal components against impacts, vibration, dust ingress, and moisture while meeting flammability standards such as UL 94 V-0 or 5 VA and biocompatibility standards applicable to medical device housings. The chassis dimensions are optimized for stationary placement proximate to the oxygen source in configurations including floor-standing placement with dimensions similar to small appliances, table-mounted or shelf-mounted positioning for elevated installation, wall-mounted configurations using brackets or keyhole slots, integrated mounting within or atop the concentrator using adapter brackets, or portable configurations with handle grips and protective bumpers for transport between locations. Exemplary implementations might measure approximately 8-12 inches in height, 6-10 inches in width, and 4-8 inches in depth, though dimensions may range more broadly from compact designs of 4×4×2 inches for minimal footprint to large clinical-grade enclosures of 16×16×8 inches accommodating expanded capabilities, providing sufficient internal volume for flow control valve assemblies with dimensions typically ranging from 2-6 inches in length, control electronics boards measuring 3-8 inches per side, touchscreen display panels with diagonal dimensions from 3-10 inches, power supply modules including AC-DC converters or battery packs, cooling systems such as fans or heat sinks when thermal dissipation requirements exceed passive cooling capacity, and connection interfaces including pneumatic fittings, electrical connectors, and cable strain reliefs while maintaining reasonable footprint for home environment placement avoiding excessive space consumption, clinical environment integration meeting infection control and space utilization requirements, or mobile configurations enabling transport in carrying cases or travel bags.

In accordance with an embodiment, the base unit includes a touchscreen interface integrated into the front panel, side panel, top panel, or adjustable arm of the chassis enabling user positioning for optimal viewing angle. The touchscreen comprises a resistive touch sensor responding to pressure from fingers, styluses, or gloved hands, a capacitive touch sensor responding to conductive contact from bare fingers or specialized styluses, a surface acoustic wave sensor detecting touch through ultrasonic wave disruption, an optical touch sensor detecting touch through infrared beam interruption, or projected capacitive technology enabling multi-touch gestures and operation through protective cover glass overlaying an LCD display providing backlighting and color capability with technologies including twisted nematic (TN), in-plane switching (IPS), or vertical alignment (VA) offering various viewing angles and color accuracy, an OLED display panel providing self-emissive pixels with high contrast ratios exceeding 100,000:1 and wide viewing angles, an e-paper display providing high visibility in bright ambient light with minimal power consumption, or LED matrix displays providing high brightness for visibility in varied lighting conditions with diagonal screen sizes ranging from 2.4 inches for compact implementations to 10 inches or larger for clinical applications where viewing from distance is important, providing sufficient display area for simultaneous presentation of multiple information elements including current physiological readings displayed in large fonts visible from distances of 3-15 feet, flow rate status shown numerically and graphically, alarm indicators with color coding and icons, time and date displays, and interactive control widgets including buttons, sliders, toggle switches, and numeric entry fields. The touchscreen resolution may range from 240×320 pixels for basic displays to 1920×1080 pixels or higher for advanced graphical interfaces supporting detailed trend plots, waveform displays, and medical imaging integration, with pixel densities from 100 to 400 pixels per inch providing sharp rendering of numerical values, graphical elements, and text labels, and sufficient contrast ratios exceeding 500:1 and brightness levels from 200 to 1000 nits for visibility under varied ambient lighting conditions ranging from darkened bedrooms at night to brightly lit clinical spaces or locations near windows with direct sunlight.

In accordance with an embodiment, the touchscreen interface software presents a primary status screen displaying the most critical real-time information in layouts optimized for at-a-glance assessment from various viewing distances and angles. The status screen may display current SpO2 percentage in large numerals with font sizes ranging from 48 to 144 points occupying a substantial portion of screen area for visibility from distances of 5 to 15 feet, heart rate in beats per minute displayed with font sizes from 36 to 96 points positioned prominently adjacent to or below the SpO2 reading, present oxygen flow rate delivery in liters per minute shown with precision from integer values to tenths or hundredths of a liter using font sizes from 36 to 96 points, and operating mode indicators showing whether the system is in automatic, manual, semi-automatic, scheduled, or learn mode through text labels, icon representations such as gear symbols for automatic mode or hand symbols for manual mode, color coding such as blue for automatic and green for manual, or combinations thereof. Additional status information may include current date and time, wireless signal strength indicators showing connection to the oximeter through bar graphs or percentage values, battery level for the wireless oximeter displayed as percentage, icon, or estimated runtime remaining, alarm status showing active alarms or time since last alarm, trend indicators showing whether SpO2 is stable, rising, or falling through arrow symbols or graphical plots, or patient identification information when multiple patients share equipment in clinical settings. The status screen layout may employ hierarchical information architecture with critical values occupying central screen regions with maximum size, supporting information positioned in peripheral regions with reduced size, alarm conditions overlaying normal displays with high-contrast highlighting, or customizable layouts enabling users to prioritize information elements based on individual preferences or clinical protocols. This status screen enables users to assess system operation at a glance from several feet away, important for caregivers checking on patients remotely from across rooms, patients verifying system status from bed or seating positions without approaching the device, clinical staff monitoring multiple patients from central nursing stations when networked displays are available, or family members ensuring proper operation without detailed technical knowledge.

In accordance with an embodiment, additional interface screens accessible through touchscreen navigation provide access to configuration parameters, historical data, diagnostic information, and system settings. Navigation may be implemented through tab interfaces with labeled tabs along screen edges, hierarchical menu systems with main menus and submenus accessed through sequential selections, swipe gestures moving between screens horizontally or vertically, dedicated navigation buttons including back, home, and menu buttons positioned consistently across screens, breadcrumb trails showing navigation path and enabling quick return to higher levels, or voice commands when speech recognition is integrated. A settings screen enables users to configure threshold values by selecting from predefined profiles such as “Conservative” (88-96%), “Standard” (90-95%), or “Aggressive” (92-98%), entering custom values through numeric keypads or slider controls with resolution from 1% increments to 0.1% increments, adjusting alarm volume through slider controls or increment buttons with levels from mute to maximum across ranges of 50-100 decibels, configuring alarm escalation timing by setting delay periods before escalation occurs ranging from 1 to 30 minutes with resolution from 1 minute to 1 second increments, setting maximum flow rate limits by entering values from 1 to 20 LPM or selecting percentages of source capacity such as 80% or 100%, configuring date and time through calendar selectors and time entry fields with automatic daylight saving adjustment or manual timezone configuration, managing wireless oximeter pairing through device discovery interfaces showing available devices with signal strength indicators and pairing buttons, configuring network connectivity including WiFi network selection with password entry and connection status display, or setting user preferences such as display brightness from 10% to 100%, screen timeout periods from 15 seconds to always-on, language selection from multiple supported languages, units selection between metric and imperial measurements, or accessibility options including high-contrast modes, large font modes, or screen reader integration.

In accordance with an embodiment, a data review screen displays historical therapy logs with multiple visualization options enabling analysis of therapy patterns over time. Graphical time-series plots may show SpO2 trends as line graphs with time on the horizontal axis spanning selectable windows including the past hour, past 4 hours, past 8 hours, past 24 hours, past week, or past month and SpO2 percentage on the vertical axis spanning ranges from 70% to 100% or dynamically scaled to data ranges with optional reference lines indicating threshold levels, target ranges shown as shaded bands, or alarm events marked with vertical lines or symbols. Flow rate adjustments may be overlaid on SpO2 plots as step functions showing flow changes over time, displayed on separate synchronized plots sharing the same time axis enabling visual correlation between flow changes and SpO2 response, or shown as bar charts indicating flow level during discrete time periods. Heart rate data may be displayed as line plots, dot plots showing individual measurements, or statistical summaries showing average, minimum, and maximum values over time windows. Alarm events may be indicated through colored markers, vertical event lines, text annotations, or separate event timelines showing alarm start and end times with duration and severity information. The data review interface may provide zooming capabilities enabling users to expand time ranges for detailed examination of specific events or compress ranges for overview of long-term trends, panning capabilities to scroll through historical data, filtering options to show only specific data types such as alarm periods or manual adjustment sessions, statistical analysis displaying average values, standard deviations, percentiles, time-in-range metrics showing percentage of time SpO2 remained within target ranges, or export functions generating reports in formats such as PDF, CSV, or graphical images suitable for sharing with healthcare providers or inclusion in medical records. A diagnostics screen displays system health information including power supply status showing AC power presence, battery charge level when backup batteries are installed, voltage levels, or charging status, wireless signal strength to the oximeter displayed as RSSI values in dBm ranging from −30 to −90 dBm, percentage indicators, or bar graphs with color coding, component self-test results showing pass/fail status for valve operation, sensor calibration, memory integrity, or communication subsystems, error logs capturing timestamped error events with codes, descriptions, and frequency counts enabling troubleshooting of intermittent issues, maintenance reminders indicating when filters require replacement, sensors need cleaning, or calibration verification is due based on elapsed time or usage metrics, or firmware version information showing installed software versions, available updates, or update history.

In accordance with an embodiment, the base unit incorporates connection interfaces for oxygen tubing attachment with pneumatic fittings designed for secure, leak-free connection and ease of use for patients with limited dexterity. A proximal oxygen inlet port receives tubing from the oxygen source including concentrators or wall outlets through connection mechanisms such as barbed fittings with hose clamps for flexible tubing with inner diameters from 3/16 to ⅜ inches, compression fittings with threaded nuts providing tool-free connection and disconnection, quick-connect fittings with push-to-connect operation and release buttons or collars for rapid tubing changes, threaded fittings using DISS, CGA, or ISO standard threads for direct connection to concentrator outlets or regulated cylinder outputs, or swivel fittings preventing tubing torsion and kinking during patient movement. A distal oxygen outlet port provides connection for patient oxygen tubing leading to the nasal cannula or oxygen mask through similar fitting types selected for compatibility with standard medical oxygen tubing and patient interfaces. These connection interfaces employ standardized medical gas fittings ensuring secure attachment and preventing disconnection during normal use through mechanical retention forces exceeding specified pull-out loads such as 5, 10, or 20 pounds-force, o-ring seals or gasket seals preventing leakage at connection points maintaining leak rates below specified limits such as 1 mL/min or 10 mL/min at operating pressures, and visual or tactile indicators such as audible clicks, indicator windows showing connection status, or colored indicators confirming proper engagement. In some embodiments, the connection interfaces include keyed or threaded designs preventing incorrect connection orientation such as reverse installation or cross-connection between inlet and outlet ports through asymmetric fitting profiles, different thread types or sizes, color coding such as green for oxygen supply connections, or labeling with directional arrows and text labels indicating “IN” and “OUT” or “SOURCE” and “PATIENT.” The connection interfaces may incorporate integrated pressure relief valves preventing over-pressurization if downstream restrictions occur, filters capturing particulate matter from oxygen supply preventing contamination of flow control components with filter elements rated for particle sizes from 5 to 50 microns, or flow restrictors limiting maximum flow under failure conditions ensuring patient safety if electronic control fails in an open state.

In accordance with an embodiment, internal to the base unit between the oxygen inlet and outlet ports resides the flow control hardware assembly positioned within the oxygen flow path and configured to regulate flow according to control signals from the co-housed controller. This assembly comprises a proportional valve such as the Festo VEMD series proportional valve, Parker Hannifin proportional valves, SMC Corporation proportional valves, Alicat mass flow controllers, Brooks Instrument mass flow controllers, or equivalent precision flow control valves from other manufacturers capable of regulating oxygen flow across the full therapeutic range from minimum flows of 0, 0.1, or 0.5 liters per minute to maximum flows of 15, 20, 25, or 30 liters per minute depending on application requirements and oxygen source capabilities. The valve receives control signals from the co-housed host controller directing the valve to maintain a specified flow rate through analog signals such as 0-5 volt, 0-10 volt, or 4-20 milliamp current loops where signal magnitude corresponds to desired flow rate with linear or non-linear transfer functions, digital signals using serial protocols such as RS-232, RS-485, Modbus RTU, or Modbus TCP transmitting flow setpoint commands as numerical values, pulse-width modulation signals where duty cycle from 0-100% corresponds to desired valve position or flow rate, or CAN bus messages containing flow setpoint data along with status requests and configuration parameters. Integral or adjacent flow sensors measure actual oxygen flow providing feedback to the host controller enabling closed-loop control that compensates for varying source pressures ranging from 5 to 60 PSI, outlet resistances varying with tubing length from 5 to 100 feet and tubing diameter, patient breathing patterns creating pulsatile flow demands, or valve performance variations due to temperature, aging, or contamination. The flow sensors may employ thermal mass flow sensing measuring gas cooling effect on heated elements, differential pressure sensing measuring pressure drop across calibrated orifices or restrictions, ultrasonic flow sensing measuring transit time differences of ultrasonic pulses traveling with and against flow, or turbine flow sensing measuring rotational speed of impellers positioned in the flow stream, providing measurement accuracy within ±0.1 to ±2% of reading or ±0.05 to ±0.5 LPM absolute error and response times from 10 milliseconds to 2 seconds enabling real-time control loop operation.

In accordance with an embodiment, the proportional valve technology enables precise incremental flow adjustments responding to automated control algorithms with resolution from 0.01 to 0.5 LPM and adjustment speeds from 0.1 to 5 LPM per second. When the automated control software determines that a flow rate increase is required due to detected desaturation with SpO2 falling below configured lower thresholds, the software commands a specific flow rate setpoint to the valve controller calculated through control algorithms including threshold-based step increases adding fixed increments such as 0.5, 1.0, or 2.0 LPM when thresholds are crossed, proportional control increasing flow by amounts proportional to the SpO2 error magnitude such as 0.2 LPM per 1% SpO2 below target, PID control implementing proportional, integral, and derivative terms to minimize steady-state error and overshoot with tunable gain parameters, model predictive control calculating optimal flow trajectories over future time horizons from 30 seconds to 5 minutes considering physiological response dynamics and constraints, or adaptive control modifying algorithm parameters based on observed patient response characteristics learned over hours to weeks of operation. The valve actuator responds within milliseconds to seconds, modulating valve position through stepper motor rotation advancing in discrete steps from 200 to 50,000 steps per revolution providing fine resolution, voice coil actuator displacement driven by proportional current providing continuous positioning, piezoelectric actuator expansion providing ultra-fast response for rapid adjustments, or servo motor control with encoder feedback providing precise position control to achieve the commanded flow rate. The flow sensor confirms achieved flow rate by measuring actual oxygen flow and transmitting measurement signals to the controller, with the control loop making fine adjustments iteratively comparing measured flow to setpoint flow, calculating flow error, applying control algorithm to determine valve position correction, commanding valve adjustment, and repeating at loop rates from 10 Hz to 1000 Hz to maintain the setpoint despite varying conditions with steady-state accuracy within ±0.05 to ±0.5 LPM and settling times from 0.5 to 3 seconds after setpoint changes.

In accordance with an embodiment, the flow control hardware incorporates a failsafe bypass valve mechanism ensuring continuous oxygen availability independent of system power status, communication connectivity, or electronic component functionality. The failsafe valve architecture addresses a critical safety requirement particularly relevant for the oxygen therapy patient population including individuals with severe respiratory compromise such as advanced COPD, pulmonary fibrosis, or congestive heart failure for whom even brief oxygen supply interruption ranging from 30 seconds to several minutes creates immediate medical emergency risk including rapid desaturation to dangerous levels below 80%, 75%, or 70% SpO2, acute dyspnea causing severe distress, syncope or loss of consciousness, cardiac arrhythmias, seizures, or cardiopulmonary arrest in extreme cases. The failsafe valve provides the redundant safety mechanism required for life-support applications by ensuring that device failure modes including power loss, electronic component failure, software faults, communication disruptions, or intentional system deactivation never result in cessation of oxygen delivery, instead gracefully degrading to manual flow control mode functionally equivalent to operation without any add-on device present.

In accordance with an embodiment, the failsafe bypass valve comprises a normally-open valve positioned in parallel with the electronically-controlled proportional valve within the pneumatic circuit topology, a normally-closed proportional valve with mechanical bypass integrated within the valve body, a three-way valve directing flow between controlled and bypass paths with failsafe positioning, or redundant valve arrays with automatic switchover upon primary valve failure. The parallel normally-open bypass valve implementation positions the bypass valve in a pneumatic path from the proximal oxygen inlet to the distal oxygen outlet that operates independently from the proportional valve path, with pneumatic check valves, flow resistors, or pressure regulators ensuring that during normal operation when the bypass valve is closed substantially all oxygen flows through the proportional valve path enabling precision regulation, while when the bypass valve opens either due to power loss or intentional activation substantially all oxygen flows through the bypass path with minimal restriction beyond that of standard oxygen tubing. In the system's unpowered state—such as during initial installation before power connection, during power failures from AC outage, circuit breaker tripping, or power supply failure, or when the system is intentionally deactivated through user shutdown command or emergency override button—the normally-open bypass valve provides an unobstructed flow path from the oxygen source to the patient oxygen line. Oxygen flows freely through the bypass valve with flow resistance comparable to straight tubing sections measuring 6-12 inches in length, resulting in pressure drops less than 0.5 to 2 PSI at typical flow rates ensuring minimal impact on effective flow delivery, with flow rate determined solely by the manual flow control setting at the oxygen source including the concentrator's built-in flowmeter dial typically offering settings from 1 to 10 LPM in 0.5 or 1.0 LPM increments or wall outlet flowmeter typically offering settings from 1 to 15 LPM, functionally identical to oxygen delivery configuration before add-on device installation.

In accordance with an embodiment, when the system powers on and initializes successfully completing self-test routines verifying valve operation, sensor functionality, microcontroller operation, memory integrity, and communication subsystem functionality within initialization periods from 2 to 30 seconds, a solenoid actuator or motorized mechanism closes the bypass valve, redirecting oxygen flow through the electronically-controlled proportional valve that provides precision regulation according to automated or manual control commands. The bypass valve remains closed during normal powered operation with all oxygen delivery passing through the controlled regulation path, maintained in closed position through continuous energization of solenoid coils with holding currents from 50 to 500 milliamps, servo motor positioning with active holding torque, latching mechanisms holding the valve closed with periodic verification pulses, or spring-preloaded closure with electromagnetic or motorized opening actuation requiring continuous power for open state. The system monitors power supply status through voltage monitoring circuits detecting AC presence and DC voltage levels remaining within acceptable ranges such as ±10% of nominal, component health through internal diagnostics including valve position verification comparing commanded and actual positions, sensor checks verifying reasonable physiological values and signal quality metrics, processor watchdog timers detecting software faults or processor lockup conditions, and communication integrity monitoring command acknowledgment and data packet reception, maintaining the bypass valve in closed position as long as these system health indicators confirm proper operation with fault detection latencies from milliseconds to seconds enabling rapid failsafe activation upon fault detection.

In accordance with an embodiment, upon power loss detected through voltage monitors sensing AC loss or DC voltage dropping below minimum thresholds such as 85% of nominal, system shutdown command issued through touchscreen interface menu selections or dedicated power buttons, detection of critical component failure through self-diagnostic routines identifying valve failures preventing flow regulation, sensor failures preventing SpO2 monitoring or flow measurement, controller failures detected through watchdog timer expiration or memory parity errors, or communication failures preventing oximeter telemetry reception for extended periods exceeding timeout thresholds from 30 seconds to 5 minutes, the bypass valve automatically returns to its normally-open state. Normally-open operation means that the valve's default mechanical position without electrical power or control signals is fully open with maximum flow area, achieved through spring mechanisms, pneumatic pressure biasing, gravity positioning, or magnetic latching in open state requiring power for closure. This fail-safe-to-open design philosophy ensures that loss of power or control immediately restores oxygen flow through the unobstructed bypass path within response times from milliseconds for spring-return solenoid valves to seconds for motorized valves with position verification. For solenoid-actuated bypass valves, spring mechanisms such as compression springs, extension springs, or torsion springs with spring constants selected to provide closing forces from 1 to 20 pounds at working displacements hold the valve open against pneumatic pressure forces and friction, with the solenoid coil generating magnetic force through electromagnetic attraction of armatures or plungers when energized to close the valve against spring pressure, requiring continuous current or pulsed holding current; loss of power de-energizes the coil, allowing spring force to open the valve within response times from 10 to 500 milliseconds depending on spring rates, moving masses, and damping mechanisms. For motorized bypass valves, spring mechanisms similarly drive the valve to open position when motor power is absent, with motor operation rotating valve elements such as ball valves, butterfly valves, or gate valves from open to closed positions through gear trains, direct drive, or belt drive mechanisms, and spring return opening the valve upon motor de-energization within response times from 0.5 to 5 seconds depending on valve size, spring energy storage, and mechanical advantage of drive mechanisms.

In accordance with an embodiment, the bypass valve's failsafe operation enables manual oxygen therapy continuation during system maintenance including filter replacement, software updates, hardware inspection, or troubleshooting procedures performed by service technicians, during troubleshooting of alarm conditions when users wish to verify oxygen delivery independent of electronic systems, or when users prefer manual control during specific activities such as sleep when automated adjustments might be perceived as disruptive, during travel when reducing system complexity is desired, or based on patient preference for direct control. A user can power off the system through the touchscreen interface by selecting shutdown menu options, confirming shutdown through dialog prompts preventing accidental shutdowns, and waiting for controlled shutdown completion including data logging finalization and valve positioning, or through dedicated hardware power buttons with press-and-hold requirements preventing accidental activation, triggering bypass valve opening occurring automatically during the shutdown sequence and returning the oxygen source to direct manual control. The patient continues receiving oxygen at the flow rate set manually at the concentrator flowmeter dial or wall outlet flowmeter knob, identical to oxygen delivery without any add-on device present in the system, providing continuity of therapy during system unavailability. When the user restores system power through touchscreen activation, power button press, or reconnection of AC power after outage restoration, the bypass valve closes automatically once the system completes self-test routines verifying all components operational within initialization periods from 2 to 30 seconds, returning operation to automated control mode resuming the operating mode active before shutdown or defaulting to safe modes such as manual mode at conservative flow rates requiring user acknowledgment before automatic control activation.

In accordance with an embodiment, the failsafe bypass valve mechanism reflects a fundamental principle in medical device design for life-critical applications: device failure or removal must never eliminate the underlying life-support function. This principle extends beyond oxygen therapy to other life-support systems including mechanical ventilation where ventilator circuit disconnection must enable spontaneous breathing, cardiac pacing where pacemaker failure must not prevent intrinsic cardiac rhythm, or renal dialysis where access disconnection must preserve vascular integrity. The bypass valve ensures that the system's universal add-on architecture truly adds functionality including automated titration, remote monitoring, data logging, and alarm capabilities without introducing new failure modes that could compromise patient safety by interrupting oxygen delivery, creating flow restrictions increasing breathing work, or causing pressure hazards through valve failures in closed positions. Users gain the benefits of automated titration maintaining optimal SpO2 during activities and remote control enabling flow adjustments without physical access to concentrators when the system operates normally, while retaining the baseline manual oxygen therapy capability that existed before system installation in any failure scenario ensuring that device availability never determines oxygen availability. This design approach contrasts with integrated titration systems requiring replacement of functional concentrators where system failure necessitates reverting to reserve concentrators if available or emergency oxygen cylinders, creating therapy interruption, equipment switching delays, and dependency on backup equipment availability.

In accordance with an embodiment, the enhanced flow capacity specification extends the therapeutic range beyond typical home oxygen concentrator outputs commonly providing maximum continuous flow rates of 5 liters per minute for basic concentrators, 10 liters per minute for standard concentrators, or 12 liters per minute for higher-end models, with higher-end models such as the Inogen At Home reaching 10 LPM, the Philips Respironics EverFlo Q reaching 5 LPM, or the AirSep VisionAire 5 reaching 5 LPM. Hospital oxygen outlets typically provide flow rates up to 15 LPM through standard flowmeters with some specialized high-flow outlets providing 25 LPM or higher. By rating the flow control hardware for 0-20 LPM capacity, 0-25 LPM capacity, or 0-30 LPM capacity depending on implementation choices, the system accommodates high-flow oxygen therapy applications where flow rates from 15 to 60 LPM are delivered through specialized high-flow nasal cannulas providing heated and humidified oxygen, ensures compatibility with high-output oxygen sources including hospital wall outlets, large clinical-grade concentrators, or manifolded cylinder systems, provides headroom for future equipment advancements as concentrator technology evolves to deliver higher flows, accommodates peak flow demands during acute desaturation events or physical exertion where instantaneous requirements may exceed resting flow rates by factors of 1.5× to 3×, and ensures that the flow control hardware does not artificially constrain therapeutic flexibility limiting clinician prescribing options or patient activity levels.

In accordance with an embodiment, high-flow oxygen therapy—distinct from conventional low-flow oxygen therapy delivered through standard nasal cannulas with flows typically from 1 to 6 LPM or simple face masks with flows from 6 to 10 LPM—employs specialized delivery interfaces providing heated and humidified oxygen at rates exceeding 15 to 20 LPM in clinical applications treating severe respiratory failure, post-operative respiratory support, or acute COPD exacerbations where conventional low-flow therapy proves insufficient. While specialized high-flow therapy systems exist such as the Vapotherm Precision Flow, Fisher & Paykel Airvo, or ResMed Optiflow incorporating integrated heating, humidification, and blending capabilities with dedicated flow generators, there are clinical scenarios where conventional concentrators supplement high-flow devices during weaning periods transitioning from high-flow to low-flow support, where providers incrementally increase flow rates beyond typical ranges for specific patient needs during activities or altitude exposure, or where economic constraints favor maximizing concentrator output rather than acquiring specialized equipment. The 0 -20 LPM rating ensures that the flow control hardware does not artificially constrain therapeutic flexibility enabling providers to optimize oxygen delivery across the full spectrum of respiratory support modalities ranging from nocturnal supplementation at 1-2 LPM, ambulatory support at 2-6 LPM, exertional support at 6-12 LPM, or urgent intervention at 12-20 LPM without encountering device limitations requiring equipment changes mid-therapy.

In accordance with an embodiment, the wide flow range capacity provides responsive margin for rapid flow increases addressing acute desaturation events. During physical exertion, patients may experience precipitous SpO2 drops—oxygen saturation falling from 96% to 84%, from 94% to 82%, or from 98% to 86% during short walking distances of 15 to 50 feet, stair climbing of 5 to 20 steps, or transfers from bed to chair—as metabolic demands increase oxygen consumption while static flow delivery remains unchanged. Rapid automated response to such desaturation requires the ability to quickly increase flow rates substantially, potentially increasing flow from 4 LPM to 8 LPM representing a doubling, from 6 LPM to 12 LPM, or from 8 LPM to 15 LPM, with adjustment velocities from 0.5 to 5 LPM per second and total adjustment times from 2 seconds to 30 seconds depending on control algorithm aggressiveness and patient response dynamics. Flow control hardware rated for maximum flows substantially above typical resting requirements—such as 20 LPM capacity when resting flow is 4-6 LPM providing 3-4×headroom—ensures that automated control algorithms have the regulatory authority to implement aggressive flow increases when patient physiology demands rapid intervention to prevent SpO2 from declining further into dangerous ranges below 85%, 80%, or 75% where acute complications including confusion, syncope, arrhythmias, or respiratory failure become increasingly likely.

In accordance with an embodiment, the precision flow control components employ thermal mass flow sensing technology, differential pressure measurement with precision orifices, ultrasonic transit-time measurement, or Coriolis force measurement to continuously monitor oxygen flow rates. Thermal mass flow sensing measures gas flow by detecting cooling of a heated sensor element positioned in the flow stream through temperature measurement or power measurement. As oxygen flows past the heated element maintained at elevated temperatures from 50 °C to 200 °C above ambient through resistive heating or thermoelectric heating, convective heat transfer cools the element proportionally to mass flow rate following heat transfer relationships depending on flow velocity, gas thermal properties, and sensor geometry. Control electronics measure the power required to maintain the element at a constant elevated temperature through closed-loop temperature control, with power consumption directly correlating to mass flow rate through calibration functions established during manufacture or field calibration, or measure the temperature difference between heated and unheated elements positioned upstream and downstream with the temperature differential correlating to flow rate. This measurement principle provides fast response times from 10 milliseconds to 2 seconds enabling real-time flow rate tracking with bandwidths from 0.5 Hz to 100 Hz, accuracy within ±0.5% to ±2% of reading across the measurement range or ±0.05 to ±0.5 LPM absolute error, and repeatability ensuring consistent measurements over extended operational life spanning years with drift rates less than 0.1% to 1% per year, minimal sensitivity to gas pressure variations allowing operation across supply pressures from 5 to 60 PSI with pressure compensation algorithms, and direct mass flow measurement independent of temperature and pressure when properly compensated enabling accurate measurement despite varying environmental conditions.

In accordance with an embodiment, the proportional valve actuation mechanism employs precision needle valves, ball valves, butterfly valves, diaphragm valves, or spool valves modulated by stepper motors, servo motors, voice coil actuators, piezoelectric actuators, or solenoid actuators. Stepper motor actuation provides open-loop positioning control advancing valve elements through discrete angular steps from 200 to 50,000 steps per revolution through bipolar or unipolar winding energization sequences, with step sizes from 1.8 degrees to 0.0072 degrees providing positioning resolution, gear reduction multiplying positioning resolution and torque output by factors from 1:1 to 100:1, and microstepping techniques subdividing full steps into microsteps from 2 to 256 microsteps per full step providing smooth motion and enhanced resolution. Voice coil actuators provide continuous positioning proportional to applied current through electromagnetic force generation between permanent magnets and current-carrying coils, enabling rapid bidirectional motion with response times under 10 milliseconds, high-resolution positioning limited only by control electronics and position sensing, and direct linear motion without mechanical conversion. Piezoelectric actuators provide ultra-compact actuation through material expansion upon voltage application, enabling nanometer-scale positioning resolution, response times under 1 millisecond, and minimal power consumption but limited displacement ranges requiring mechanical amplification. The control electronics receive flow rate setpoint commands through various interfaces and modulate valve position to achieve the commanded flow through control algorithms. The thermal mass sensor or other flow sensor provides continuous flow rate feedback measuring actual flow, with the control loop making rapid position adjustments comparing measured flow to setpoint flow, calculating flow error, applying PID control or other algorithms to determine actuator drive signal, commanding actuator motion, and repeating iteratively maintaining the setpoint despite disturbances including source pressure variations, outlet restriction changes, or valve performance drift. Response times from setpoint change to achieved flow stabilization typically occur within 0.5 to 5 seconds for thermal mass controllers, 0.1 to 2 seconds for voice coil systems, or under 0.5 seconds for piezoelectric systems, fast enough for responsive automated adjustment to desaturation events without creating jarring discontinuities in oxygen delivery that might be perceptible to patients through pressure changes or breathing effort variations.

In accordance with an embodiment, the system architecture supports modular enhancement for future iterations through communication protocol design and software architecture planning. The underlying technical architecture enables subsequent integration of mobile applications providing smartphone or tablet interfaces, desktop workstation interfaces for clinical staff, hospital EHR system connectivity exchanging patient data and receiving orders, advanced analytics features including machine learning algorithms, telemedicine integration enabling remote provider consultation, or Internet of Things (IoT) integration connecting to home automation systems without requiring redesign of core flow control and safety mechanisms validated during initial regulatory approval. The modular enhancement capability stems from several architectural design choices including layered software architecture separating application logic from hardware control, abstraction interfaces defining clear boundaries between components, standardized communication protocols enabling interoperability, and extensible data structures accommodating future parameter additions.

In accordance with an embodiment, the communication system employs standardized protocols rather than proprietary communication schemes. Bluetooth Low Energy for oximeter wireless telemetry follows Bluetooth Special Interest Group specifications ensuring interoperability with smartphones, tablets, and computers manufactured by diverse vendors, enabling development of companion mobile applications using standard BLE APIs available in iOS, Android, Windows, and Linux operating systems without requiring proprietary protocol reverse engineering. TCP/IP networking protocols for future network connectivity provide universal connectivity to local area networks and internet enabling cloud services, remote monitoring, and system updates through standard infrastructure. These standard protocols provide well-documented interfaces published in freely available specifications, software development kits provided by protocol organizations and hardware vendors, and extensive developer communities providing support resources enabling development of additional software applications that communicate with the base unit. For example, a mobile application development project can utilize standard BLE service discovery scanning for advertised services and characteristics, characteristic subscription registering for notifications of measurement updates, and characteristic writes transmitting control commands to receive real-time telemetry from the base unit and send control commands, with no modifications required to base unit firmware beyond exposing appropriate BLE services and characteristics during development.

In accordance with an embodiment, the data logging format uses structured data representations enabling external applications to parse and analyze logged therapy data. Logged data may be stored in comma-separated values (CSV) format with header rows defining column labels and data rows containing timestamped measurements, JavaScript Object Notation (JSON) format with hierarchical structures representing measurement objects with properties and values, Extensible Markup Language (XML) format following healthcare data schemas, binary formats with documented structure specifications optimizing storage efficiency, or database formats such as SQLite enabling structured queries that standard analysis tools readily import. Healthcare providers or researchers can transfer logged data files to computers through USB connections, wireless transmission, or cloud synchronization and load them into spreadsheet applications such as Microsoft Excel, Google Sheets, or LibreOffice Calc for tabular analysis and charting, statistical analysis software such as R, Python with pandas/numpy/scipy libraries, or MATLAB for advanced statistical modeling and hypothesis testing, custom visualization tools developed in web technologies, desktop applications, or specialized medical data viewers, or machine learning platforms such as TensorFlow, PyTorch, or scikit-learn for pattern recognition and predictive modeling for detailed examination without dependency on proprietary data access mechanisms requiring vendor-specific software or licensing.

In accordance with an embodiment, the control command structure implements abstraction layers separating high-level control intentions from low-level hardware actuation details. External applications or interfaces need only send high-level commands such as “set flow rate to 8 LPM” specifying desired therapeutic effect, “configure lower SpO2 threshold to 90%” specifying clinical parameters, “activate alarm silence for 5 minutes” specifying user intentions, or “export data for date range” specifying information retrieval without concerning themselves with proportional valve positioning algorithms translating flow setpoints to valve positions considering valve characteristics and calibration, sensor calibration coefficients compensating for sensor drift or environmental effects, communication packet formatting assembling data into protocol-compliant structures, or error recovery mechanisms handling transient communication failures. The base unit firmware implements these hardware control details internally within abstraction layers presenting simplified control interfaces to external systems through application programming interfaces (APIs), command sets, or service-oriented architectures. This abstraction enables future enhancements to flow control mechanisms—such as upgrading to different valve models with different control characteristics, adding redundant flow sensors requiring sensor fusion algorithms, or implementing advanced control algorithms with additional tuning parameters—without breaking compatibility with existing control software developed for earlier system versions, ensuring that mobile applications, hospital integrations, and third-party extensions continue functioning across product generations without requiring synchronous updates to all system components.

In accordance with an embodiment, deployment of core safety and automation features in initial implementations maintains clear migration paths for advanced capabilities through forward compatibility in data structures and protocols. Early adopters receive functional automated oxygen therapy management through touchscreen base unit interfaces addressing immediate patient needs for movement independence through wireless monitoring and automated adjustment, safety enhancement through alarm systems and failsafe mechanisms, and treatment optimization through data logging enabling provider review. Clinical validation studies using these initial implementations provide empirical data demonstrating efficacy through measured outcomes such as reduction in desaturation event frequency or duration, safety through adverse event monitoring and failure mode analysis, usability through user satisfaction surveys and human factors testing, and reliability through field performance monitoring informing regulatory submission materials with clinical evidence and building user confidence in the technology through published results, clinical testimonials, and word-of-mouth adoption.

In accordance with an embodiment, subsequent product iterations add mobile application interfaces providing smartphone-based monitoring and control. Mobile applications may be developed for iOS platforms requiring App Store approval and distribution, Android platforms distributed through Google Play or direct installation, cross-platform frameworks such as React Native or Flutter enabling single codebase deployment to multiple platforms, or progressive web applications accessible through mobile browsers without installation offering convenience for patients and caregivers checking status remotely within the home or while away from home monitoring status through internet connectivity, receiving notifications when alarm conditions occur outside audible range of the base unit through push notification services provided by mobile operating systems, accessing historical data and trends through mobile-optimized graphical interfaces, or making control adjustments when authorized by care protocols such as temporary flow increases before anticipated activities. The mobile application connects to the base unit via WiFi direct connections when on the same local network, Bluetooth connections for proximity-based control within 30-300 feet, or internet-based connections through cloud relay services enabling remote monitoring from unlimited distances when both base unit and mobile device have internet connectivity, subscribing to telemetry streams receiving SpO2, heart rate, flow rate, and alarm status updates, and sending control commands using the same underlying protocols and abstraction layers that the touchscreen interface uses internally. No changes to core flow control or safety algorithms are required; the mobile application provides an alternative user interface accessing existing functionality through standardized communication channels implemented in the base unit firmware.

In accordance with an embodiment, further iterations integrate hospital EHR systems using HL7 messaging or FHIR APIs. The base unit establishes network connectivity to hospital information systems through wired Ethernet connections, secure WiFi connections to hospital networks, or VPN tunnels through internet connections for remote facilities, exchanging ADT (Admission, Discharge, Transfer) messages associating flow control systems with current patient assignments enabling automatic configuration when patients are admitted or transferred, transmitting therapy logs as structured clinical observations formatted as HL7 ORU messages or FHIR Observation resources containing timestamped SpO2, heart rate, flow rate, and alarm data with patient identifiers and device identifiers for integration into patient timelines and flowsheets, and receiving protocol configuration parameters from physician orders entered in computerized physician order entry (CPOE) systems specifying oxygen therapy prescriptions including target SpO2 ranges, maximum flow limits, monitoring requirements, or alarm criteria for automatic configuration of device parameters eliminating manual transcription and reducing configuration errors. This hospital integration leverages the data logging and threshold configuration mechanisms already present in the base unit, extending their interfaces to accept input from hospital databases through inbound message processing and provide output to hospital databases through outbound message generation rather than only local touchscreen interaction, with message transformation handling format conversions, data mapping between device-specific and standard terminologies, and workflow orchestration coordinating multi-step processes.

In accordance with an embodiment, advanced analytics features implement machine learning algorithms analyzing historical therapy data to predict desaturation events enabling preventive flow increases, optimize threshold parameters automatically learning patient-specific response characteristics, detect anomalies indicating equipment issues or patient condition changes, or personalize treatment protocols based on individual patterns. These algorithms process the second-by-second time-series data already being logged including SpO2, heart rate, flow rate, activity levels, and environmental factors creating feature vectors for machine learning models. The development pathway starts with offline analysis of exported log files from multiple patients building predictive models through supervised learning training neural networks, decision trees, or support vector machines on labeled datasets identifying desaturation events or equipment failures, unsupervised learning clustering algorithms identifying patient subgroups with similar patterns, or reinforcement learning agents learning optimal control policies through simulated or real-world interaction, then progresses to deploying trained models within the base unit firmware for real-time predictive adjustment executing models on embedded processors or uploading models to cloud services for inference with results transmitted back to devices or hybrid edge-cloud approaches where preliminary processing occurs locally with detailed analysis in cloud. The consistent data logging infrastructure across product iterations ensures that insights gained from initial deployment data inform subsequent algorithmic enhancements, with iterative improvement cycles collecting data from fielded devices, refining models with expanded datasets, validating improvements through A/B testing or clinical trials, and deploying updates through over-the-air firmware updates or cloud-based model updates.

In accordance with an embodiment, medical device development involves regulatory pathways including FDA 510(k) premarket notification demonstrating substantial equivalence to predicate devices, De Novo classification for novel low-to-moderate risk devices without predicates, Premarket Approval (PMA) for high-risk devices requiring clinical trials, or international pathways such as CE marking in Europe requiring conformity assessment to Medical Device Regulation (MDR), clinical validation through feasibility studies, pilot studies, or pivotal trials demonstrating safety and efficacy, and manufacturing scale-up establishing quality systems compliant with ISO 13485, FDA Quality System Regulation (QSR), or equivalent standards. By focusing initial regulatory submissions on core safety and efficacy features with mature component technologies—off-the-shelf wireless pulse oximeters with established performance and regulatory history, proven proportional valve flow controllers used in industrial or medical applications, and established touchscreen HMI platforms meeting medical device usability standards—the path to regulatory clearance or approval is streamlined compared to submissions incorporating cutting-edge mobile health applications requiring validation of novel user interfaces and connectivity scenarios, cloud-based analytics platforms requiring cybersecurity validation and data privacy compliance, or artificial intelligence algorithms requiring explanation of training data, model validation, and bias assessment imposing additional validation burden, time to market, and regulatory risk.

In accordance with an embodiment, via mechanisms understood in the art, enhancements roll out through software updates to the base unit firmware delivered through USB connections during service visits, wireless over-the-air updates when internet connectivity is available, or automatic updates in clinical environments with centralized device management and companion application releases distributed through app stores, web downloads, or automatic application updates. Users benefit from continuous improvement without requiring hardware replacement gaining new features such as enhanced algorithms, additional connectivity options, or refined user interfaces, bug fixes addressing issues identified through field use and user feedback, performance optimizations improving response times or battery life, or security updates addressing newly identified vulnerabilities maintaining device security over product lifetime, analogous to consumer electronics products that gain features through software updates over their operational lifetimes such as smartphones receiving operating system updates for multiple years or smart home devices gaining integration with new services. The universal add-on architecture's modularity extends not only across diverse oxygen sources such as home concentrators, portable concentrators, and hospital wall outlets and patient populations including home care, long-term care, and acute care settings, but across time through extensible software architectures supporting long-term product evolution from initial feature-focused versions to mature feature-rich platforms enabling decade-scale product lifecycles with sustained clinical relevance and competitive positioning.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

I claim:

1. A system for controlling oxygen flow delivery to a patient, comprising:
a flow control hardware device configured as a universal retrofittable add-on to connect between an oxygen source and a patient oxygen line, wherein the flow control hardware device comprises:
a proximal connection interface configured to attach to an outlet of the oxygen source through standardized medical gas fittings including DISS threaded connections, Quick-Connect adapters, barbed hose fittings, or compression fittings, without requiring invasive modification of internal oxygen source components;
a distal connection interface providing an oxygen outlet configured to attach to the patient oxygen line through standardized patient interface fittings compatible with nasal cannulas, oxygen masks, and medical tubing standards;
precision flow regulation components positioned between the proximal and distal connection interfaces, said components comprising electronically-controlled valves, mass flow controllers, or proportional solenoid assemblies rated for continuous operation across a flow range of 0-20 liters per minute;
flow measurement sensors providing real-time flow rate feedback for closed-loop regulation;
wherein the flow control hardware device regulates oxygen flow from a maximum output of the oxygen source down to a specified flow rate determined by control signals, and wherein said regulation occurs external to the oxygen source preserving native source functionality;
an oximeter device configured to monitor blood oxygen concentration and heart rate of the patient, wherein the oximeter device comprises:
a finger-mountable housing dimensioned to accommodate a patient's fingertip;
a photodetector assembly comprising red and infrared light-emitting diodes and a photodiode positioned to detect light transmission through tissue;
signal processing circuitry calculating SpO2 percentage and pulse rate from detected optical signals;
a display screen rendering current physiological measurements in real-time;
a battery power supply enabling wireless operation independent of wired power connections; and
a wireless communication module transmitting measurement data to external devices;
a remote control interface integrated within the oximeter device, wherein integration comprises:
physical buttons, capacitive touch sensors, or touchscreen interface elements incorporated into the oximeter housing;
user interface software presenting flow rate control options on the oximeter display;
command generation circuitry encoding user inputs into control command data packets; and
wherein the remote control interface enables patients to adjust oxygen flow rates through direct interaction with the oximeter device worn on their finger, providing zero-latency control without requiring physical access to the oxygen source or separate control devices; and
a communication system enabling wireless transmission of control signals between the oximeter device and the flow control hardware device, wherein the communication system comprises:
a first wireless transceiver integrated within the oximeter device;
a second wireless transceiver integrated within or communicatively coupled to the flow control hardware device;
wireless communication protocols including Bluetooth Low Energy, WiFi, or other digital radio protocols operating in unlicensed frequency bands;
data packet structures encoding SpO2 measurements, heart rate data, and flow rate control commands;
security protocols including encryption and authentication mechanisms protecting data transmission; and
error detection and automatic retransmission protocols ensuring reliable command delivery;
wherein the system is configured to automatically adjust oxygen flow through the flow control hardware device in response to blood oxygen concentration measurements from the oximeter device by:
receiving wireless telemetry of SpO2 measurements at the flow control hardware device or associated control processor;
comparing received SpO2 values to predetermined threshold values configured for the patient;
generating flow rate adjustment commands when SpO2 falls below lower threshold values or exceeds upper threshold values;
transmitting adjustment commands to the flow control hardware device; and
actuating the precision flow regulation components to increase or decrease oxygen flow accordingly.

2. The system of claim 1, wherein the oxygen source comprises one of:
an oxygen concentrator device selected from the group consisting of:
stationary home oxygen concentrators rated for continuous operation at flow rates between 1-10 liters per minute;
portable oxygen concentrators utilizing pressure swing adsorption or membrane separation technologies;
high-output concentrators rated for flow rates up to 20 liters per minute for clinical applications; and
pulse-dose oxygen concentrators delivering bolus oxygen volumes synchronized to patient inhalation; or
a medical gas outlet in a clinical environment comprising:
wall-mounted Quick-Connect or threaded outlet fittings connected to centralized medical gas pipeline systems;
ceiling-mounted service columns incorporating medical gas outlets along with electrical and data connections;
bedside console units integrating medical gas outlets with patient monitoring equipment; and
portable cylinder manifold systems in emergency response or transport settings;
wherein the flow control hardware device comprises universal connection interfaces enabling attachment to any of said oxygen source types without requiring source-specific adaptation or internal modification of the source equipment.

3. The system of claim 1, further comprising automated control software configured to:
continuously monitor blood oxygen concentration measurements from the oximeter device by:
receiving wireless telemetry data packets at intervals of one second or less;
parsing SpO2 values, heart rate data, and signal quality metrics from received packets;
buffering measurements in time-series data structures enabling temporal pattern analysis;
compare the measurements to predetermined thresholds by:
evaluating whether current SpO2 measurements fall below a configured lower threshold value;
evaluating whether current SpO2 measurements exceed a configured upper threshold value;
implementing moving average filters over 15-30 second windows to reduce sensitivity to transient measurement artifacts;
applying hysteresis bands to threshold comparisons preventing rapid oscillation between adjustment states; and
automatically trigger flow rate adjustments through the flow control hardware device when measurements fall outside the predetermined thresholds by:
calculating flow rate increment magnitude based on the degree of SpO2 deviation from threshold values, wherein larger deviations trigger proportionally larger flow increases;
implementing rate-limiting controls preventing flow rate changes exceeding predetermined maximum adjustment velocities to avoid overshoot;
transmitting digitally-signed flow adjustment commands to the flow control hardware via secure wireless channels;
verifying command execution through receipt of acknowledgment messages from the flow control hardware;
monitoring subsequent SpO2 measurements to assess effectiveness of adjustments; and
iteratively repeating adjustment cycles until SpO2 stabilizes within the predetermined threshold range or maximum flow rate limits are reached.

4. The system of claim 1, wherein the oximeter device comprises:
a display interface showing:
current oxygen saturation levels rendered as numerical percentage values with refresh rates of at least once per second;
heart rate displayed in beats per minute with real-time updating;
oxygen flow settings indicating current delivery rate in liters per minute received via wireless telemetry from the flow control hardware;
battery charge status for the wireless oximeter unit;
wireless connectivity status indicators showing signal strength and connection state; and
alarm condition notifications including visual indicators when SpO2 falls outside configured thresholds; and
manual control inputs enabling direct adjustment of oxygen flow rates, said inputs comprising:
physical button interfaces including increment and decrement controls for stepwise flow rate adjustment;
capacitive touch sensors enabling slide-based or tap-based control gestures;
mode selection controls toggling between automated threshold-based control and manual override;
confirmation mechanisms requiring deliberate user action to prevent accidental adjustments; and
wherein the manual control inputs transmit flow adjustment commands wirelessly to the flow control hardware device, enabling patients to modify their oxygen delivery while the oximeter remains on their finger, thereby providing zero-latency control without requiring access to the base unit or oxygen source.

5. The system of claim 1, wherein the communication system comprises:
wireless communication modules supporting multiple protocols including:
a Bluetooth Low Energy (BLE) transceiver operating in accordance with Bluetooth 5.0 or later specifications, configured for low-power communication with the oximeter device at distances up to 100 meters;
a WiFi radio supporting IEEE 802.11ac or later standards, configured for connection to facility wireless networks enabling cloud-based data synchronization and remote monitoring;
a cellular modem supporting LTE or 5G protocols, configured to provide backup connectivity when local network infrastructure is unavailable;
signal processing hardware for converting control commands into flow adjustments, said hardware comprising:
a microcontroller unit executing real-time operating system software for command parsing and validation;
analog-to-digital converters sampling sensor inputs at sampling rates sufficient for closed-loop control;
digital-to-analog converters or pulse-width modulation outputs commanding proportional valve actuators; and
watchdog timers and error detection circuits ensuring system responsiveness and detecting communication failures; and
security features for protecting transmission of patient data, said features comprising:
end-to-end encryption of wireless communications using AES-256 or equivalent cryptographic algorithms;
mutual authentication between system components using certificate-based public key infrastructure;
intrusion detection monitoring analyzing communication patterns for unauthorized access attempts;
audit logging recording all data access and control command transmissions with tamper-evident cryptographic hashing; and
compliance mechanisms ensuring conformance with HIPAA Security Rule requirements for electronic protected health information.

6. The system of claim 1, further comprising integration capabilities with hospital management systems, said integration enabling:
receipt of telemetry data from patient monitoring devices distributed throughout clinical facilities, wherein receipt comprises:
establishing network connectivity with bedside monitors, central monitoring stations, and medical device integration platforms through HL7 interface engines;
receiving ADT (Admission, Discharge, Transfer) messages to associate flow control systems with current patient assignments;
subscribing to continuous vital sign data streams from monitoring equipment assigned to specific patient beds;
control access from clinical workstations located at nursing stations, physician offices, and mobile computing devices, wherein control access comprises:
providing web-based user interfaces accessible through hospital intranet browsers requiring multi-factor authentication;
enabling authorized clinicians to view real-time SpO2, heart rate, and current flow rate for multiple patients simultaneously through centralized dashboards;
permitting remote manual adjustment of flow rates and threshold parameters through the clinical workstation interfaces with actions logged against clinician credentials; and
automated flow adjustments based on electronic medical records system data, wherein adjustments comprise:
retrieving patient-specific oxygen therapy protocols from structured data fields in the EHR;
automatically configuring threshold parameters and maximum flow rate limits based on physician orders documented in the EHR;
triggering flow rate modifications in response to clinical events recorded in the EHR including pre-operative preparation protocols, post-operative recovery pathways, and changes in respiratory therapy orders; and
documenting all automated adjustments as clinical observations within the EHR using FHIR Observation resources linked to the originating oxygen therapy orders.

7. A method for controlling oxygen delivery to a patient, comprising:
continuously monitoring blood oxygen concentration and heart rate through an oximeter device worn on a patient's finger, wherein monitoring comprises:
detecting pulsatile blood flow through photodetector sensing of light absorption at red and infrared wavelengths;
calculating oxygen saturation percentage (SpO2) based on the ratio of absorption at said wavelengths;
measuring pulse rate from the frequency of detected pulsatile waveforms; and
sampling SpO2 and heart rate measurements at a rate of at least once per second to enable rapid detection of desaturation events;
wirelessly transmitting measurement data from the oximeter device to a flow control hardware device through a battery-powered communication module incorporated within the oximeter housing, wherein transmission comprises:
encoding SpO2, heart rate, and signal quality metrics into standardized data packets;
transmitting packets via Bluetooth Low Energy protocols operating in the 2.4 GHz ISM band;
maintaining continuous wireless connection through automatic reconnection protocols when connectivity is interrupted;
comparing measurements to predetermined threshold values configured by healthcare providers, wherein comparison comprises:
maintaining in memory a lower SpO2 threshold value below which automated flow increases are triggered;
maintaining an upper SpO2 threshold value above which automated flow decreases are triggered;
continuously evaluating whether current SpO2 measurements fall outside the threshold range; and
implementing hysteresis algorithms to prevent rapid oscillation between flow adjustment states;
automatically adjusting oxygen flow through the flow control hardware device when measurements fall outside the thresholds by:
calculating required flow rate change magnitude based on the degree of SpO2 deviation from threshold values;
transmitting flow adjustment commands from automated control software to flow control hardware;
actuating precision flow regulation components to incrementally increase or decrease oxygen delivery; and
monitoring subsequent SpO2 measurements to verify effectiveness of adjustments; and
enabling manual flow rate adjustments through a control interface integrated within the oximeter device by:
detecting user interaction with physical buttons or capacitive touch sensors on the oximeter housing;
transmitting manual adjustment commands via the wireless communication link; and
overriding automated control temporarily while manual adjustment mode is active.

8. The method of claim 7, further comprising:
receiving telemetry data from hospital patient monitoring systems including bedside vital sign monitors, ventilators, and continuous monitoring equipment through standardized medical device communication protocols including IEEE 11073 and vendor-specific APIs;
processing the telemetry data through an electronic medical records system by:
aggregating physiological parameters from multiple monitoring devices associated with a single patient record;
applying clinical decision support rules comparing current vital signs against protocol-defined intervention criteria;
generating automated flow adjustment recommendations based on integrated analysis of SpO2, heart rate, respiratory rate, blood pressure, and documented patient conditions; and
automatically adjusting oxygen flow rates through the flow control hardware device based on the processed telemetry data by:
transmitting digitally-signed control commands from the hospital management system to the flow control hardware via secure authenticated channels;
implementing graduated flow rate increases when integrated vital sign analysis indicates respiratory distress patterns;
coordinating oxygen delivery adjustments with ventilator settings and medication administration records to optimize comprehensive respiratory support; and
maintaining audit trails documenting the clinical data inputs and algorithmic logic underlying each automated adjustment for regulatory compliance and quality assurance review.

9. The method of claim 7, further comprising:
maintaining detailed electronic logs of all flow rate adjustments in non-volatile memory storage, wherein each log entry comprises a timestamp, pre-adjustment flow rate, post-adjustment flow rate, trigger source identifier distinguishing between automated threshold-triggered adjustments and manual user-initiated adjustments, and contemporaneous SpO2 and heart rate measurements;
integrating the flow adjustment logs with electronic medical records systems through standardized healthcare communication protocols by:
formatting log data as HL 7 Version 2.x messages or FHIR Observation resources;
transmitting formatted data to hospital information systems via secure network connections utilizing TLS encryption; and
associating log entries with patient identification records through Medical Record Number (MRN) or other unique patient identifiers;
generating configurable alerts when oxygen saturation levels remain outside predetermined desired ranges for threshold duration periods, wherein alert generation comprises:
monitoring continuous SpO2 measurements over a sliding time window of 5-10 minutes;
comparing the percentage of time within the window that SpO2 remains below lower threshold or above upper threshold against alert activation criteria;
triggering multi-channel notifications through audible alarms at the base unit, push notifications to mobile devices of designated healthcare providers, and automated messages to hospital nurse call systems when alert criteria are satisfied; and
wherein the alerts distinguish between transient desaturation events resolved by automated flow adjustments and persistent desaturation requiring clinical intervention.

10. The system of claim 1, wherein:
the oxygen source comprises a medical gas outlet mounted in a wall structure of a clinical facility and connected to a centralized oxygen supply system;
the flow control hardware device comprises specialized connection interfaces configured to attach to standardized medical gas outlet specifications including Quick-Connect, Ohmeda, Puritan Bennett, and DISS threaded fittings;
the flow control hardware regulates high-pressure hospital oxygen supply from centralized systems operating at 50-55 PSI down to therapeutic flow rates appropriate for patient delivery through standard nasal cannula or oxygen masks;
the communication system enables integration with hospital monitoring infrastructure through secure facility network connections utilizing TCP/IP protocols over Ethernet or WiFi; and
wherein the system maintains operational consistency whether the oxygen source comprises a standalone concentrator in a home environment or a wall-mounted medical gas outlet in a hospital room, enabling deployment flexibility across diverse care settings while preserving identical flow regulation, automated adjustment, and monitoring capabilities.
